(19) United States
(12) Patent Application Publication
(10) Pub. No.: US 2016/0368980 A1
(43) Pub. Date: Dec. 22, 2016

(54) INHIBITION OF GRANULOCYTE COLONY STIMULATING FACTOR IN THE TREATMENT OF CANCER

(71) Applicant: STC.UNM, Albuquerque, NM (US)

(72) Inventors: Katherine Theresa Morris, Albuquerque, NM (US); Ellen Janine Beswick, Albuquerque, NM (US)

(21) Appl. No.: 14/832,031

(22) Filed: Aug. 21, 2015

Related U.S. Application Data
(60) Provisional application No. 62/041,477, filed on Aug. 25, 2014.

(51) Int. Cl. C07K 16/24; C07K 16/28; A61K 39/395; A61K 45/06

(52) U.S. Cl. CPC C07K 16/243; A61K 45/06; C07K 16/2866; A61K 39/3955; C07K 2317/76

(57) ABSTRACT

The present invention relates to the discovery that granulocyte colony stimulating factor (G-CSF) as well as granulocyte colony stimulating factor receptor (G-CSFR) are highly expressed in cancer tissue and the inhibition of G-CSF using a G-CSF inhibitor, which term includes inhibitors of the G-CSF receptor, represents a viable approach to the treatment of cancer, including drug resistant cancers, metastatic cancers and recurrent cancers. G-CSF inhibitors as described herein may be used alone or in combination with an at least one additional anti-cancer agent for the treatment of cancer.

### CROSS-REFERENCE TO RELATED APPLICATION

This application claims the benefit of priority of U.S. Provisional Patent Application No. 62/041,477 filed 25 Aug. 2014, entitled “Inhibition of Granulocyte Colony Stimulating Factor in the Treatment of Cancer”, the entire contents of which are incorporated by reference herein.

### STATEMENT REGARDING FEDERALLY SPONSORED RESEARCH OR DEVELOPMENT

Thisinvention was made with government support under grant no. 8UL1TR000041 awarded by the National Institutes of Health. The government has certain rights in the invention.

### FIELD OF THE INVENTION

The present invention relates to the discovery that granulocyte colony stimulating factor (G-CSF) as well as granulocyte colony stimulating factor receptor (G-CSFR) are highly expressed in cancer tissue and the inhibition of G-CSF using a G-CSF inhibitor, which term includes inhibitors of the G-CSF receptor, represents a viable approach to the treatment of cancer, especially malignant tumors, including drug resistant cancers, metastatic cancers and recurrent cancers. G-CSF inhibitors as described herein may be used alone or in combination with an at least one additional anti-cancer agent for the treatment of cancer.

### BACKGROUND AND DISCUSSION OF THE INVENTION

G-CSF is a pro-inflammatory cytokine with the well-studied function of inducing differentiation and mobilization of neutrophils. However, some tumors also express G-CSF and G-CSF receptor (G-CSFR) (Hirai et al., 2001; Ninci et al., 2000; Sunaga et al., 2001). Of concern is that activation of G-CSFR is known to stimulate common proliferation associated pathways such as MAPK and JAK/STAT (Marino and Roguin, 2008; Sampson et al., 2007), and thus the effects of G-CSFR expression on tumors should be considered. One group has demonstrated that G-CSF stimulates proliferation and migration of head and neck squamous cell carcinoma cell lines (Gutschalk et al., 2006). In addition, there are reports of highly aggressive solid tumors ranging from bladder to gastric cancers that secrete excessive G-CSF. These studies have shown that high levels of tumor-produced G-CSF are associated with poor patient outcomes (Tachibana and Murai, 1998; Yokoyama et al., 2005), and that G-CSF may stimulate tumor growth and progression.

Recently, research on the involvement of stromal cells in the initiation and development of GI cancers has progressed, and it is now acknowledged that fibroblasts/myofibroblasts (MF or α-SMA positive, CD90⁺ stromal fibroblasts) play a critical role in the initiation, growth, and metastasis (Worthley et al., 2010). These cells are thought to facilitate tumor growth and stromal invasion of tumor cells by releasing pro-metastatic factors that act on tumors in a paracrine manner. One study suggests that human fibroblasts can produce G-CSF (Seelentag et al., 1989), but the ability of tumor-associated MF to produce G-CSF has not been examined. Since MF are key regulators of chronic inflammation, tumor growth, and metastasis, we investigated whether G-CSF is highly produced by these cells in the tumor microenvironment leading to a potential role in tumor growth and development.

Since the most well-known function of G-CSF is to mobilize bone marrow derived stem cells, it is important to examine its effects on tumor cells with stem-like properties. Tumor stem marker-expressing cells are thought to be initiators of tumor growth, inducers of metastasis, and a cause of chemotherapy resistance. In gastric and colon tumors, several studies have shown that one marker indicating a stem-like population is CD44 (Wang et al., 2011; Ohata et al., 2012; Su et al., 2011a). These studies further suggest that CD44 is not only a marker of cancer initiating cells, but also of invasive cells. The enzyme aldehyde dehydrogenase has also proved useful for identification of cancer stem cells for epithelial cancers since cells that are high expressers of aldehyde dehydrogenase usually correlate with expression of other stem markers, metastasis, and poor clinical outcome (Deng et al., 2010; Sullivan et al., 2010). One study has also demonstrated that there is a direct correlation between expression of this enzyme and colon stem cells that transition from colitis to cancer, thereby demonstrating the importance of this marker in colon cancer (Carpentino et al., 2009). Since G-CSF is known to increase proliferation and mobilization of bone marrow derived hematopoietic stem cells (Liongue et al., 2009), a possible role of G-CSF in growth and mobilization of stem-like cancer cells expressing aldehyde dehydrogenase and CD44 was also examined by our lab.

Results of our work has indicated that G-CSFR is highly expressed in approximately 90% of human gastric and colorectal tumors examined. Tumor-derived stromal fibroblasts/myofibroblasts also produce increased G-CSF. Treatment of gastric and colorectal carcinoma cells with G-CSF or MF supernatants increased their proliferation and migration. Further, G-CSF expanded a stem-like subpopulation of carcinoma cells. These processes were found to be dependent on ERK1/2 and RSK phosphorylation. Our findings led us to consider whether G-CSF could be a therapeutic target for solid tumors that over express the receptor.

### BRIEF DESCRIPTION OF THE INVENTION

The inventors have recently demonstrated a significant decrease in tumor burden in mice treated with both a tumor induction model and antibody to G-CSF. The significant results obtained could not have been predicted by the existing literature, and the hypothesis that G-CSF inhibition will be an effective treatment in patients with solid tumors is a new concept for which the present application also has been filed.

The inventors have thus discovered that G-CSF and G-CSFR are up regulated in numerous cancer tissues and because of this excessive up regulation, are excellent targets for the inhibition and/or treatment of cancer using a G-CSF inhibitor, including a G-CSF inhibitor which can inhibit G-CSF receptors. From these studies the inventors have determined that G-CSF inhibitors are particularly potent anti-cancer compounds and may be used alone or in combination for the treatment and/or inhibition (including remission) of the growth, elaboration, metastasis and/or recurrence of cancer in a patient in need.

The present invention provides the bases for novel and clinically-significant therapies that supplement and complement known anti-cancer regimens.

In a first embodiment, the present invention is directed to a method of treating cancer in a patient or subject in need comprising co-administering to the patient subject a pharmaceutically effective amount of:
(a) one or more compounds which is a G-CSF inhibitor, optionally,
(b) at least one additional anticancer agent, wherein the administration of the G-CSF inhibitor and optional additional anticancer agent is optionally combined with radiation and/or other alternative therapy (e.g., hormonal therapy, proton therapy, cryosurgery, and/or high intensity focused ultrasound (HIFU), radiofrequency ablation, microwave ablation, transarterial therapies such as radioembolizationn with Y90 or bland embolization and chemoembolization (for liver cancer)) of said cancer.

In certain embodiments according to the present invention the G-CSF inhibitor(s) are administered to the cancer patient with at least one additional anticancer agent to provide a synergistic effect in the treatment of cancer.

In certain embodiments, the G-CSF inhibitor is administered in effective amounts alone or in combination with an effective amount of an additional anticancer agent as otherwise described herein for the treatment of cancer, which treatment method may be optionally combined with radiation therapy.

Related pharmaceutical formulations pursuant to the present invention are also provided.

In a particular embodiment, the present invention provides a method of treating a subject who suffers from a cancer as described herein, preferably a cancer selected from the group consisting of breast cancer, ovarian cancer, lung cancer, colorectal cancer, glioblastoma multiform (GBM), melanoma, glioma, esophageal cancer, gastric cancer, hepatocellular cancer, gallbladder cancer, cholangiocarcinoma cancer, prostate cancer, cervical cancer, uterine cancer, sarcomas, renal cancer, bladder cancer and pancreatic cancer, the method comprising co-administering to the subject a pharmaceutically-effective amount of:
(a) one or more G-CSF inhibitor compounds;
(b) one or more anticancer agents (often, a chemotherapeutic agent),
   wherein the administration of said G-CSF inhibitor(s) and said additional anticancer agent is optionally further combined with radiation or other alternative therapy of said cancer (e.g., hormonal therapy, proton therapy, cryosurgery, and/or high intensity focused ultrasound (HIFU), radiofrequency ablation, microwave ablation, transarterial therapies such as radioembolizationn with Y90 or bland embolization and chemoembolization (for liver cancer).

In certain embodiments, the subject is treated concomitantly by radiotherapy or other alternative therapy and the G-CSF inhibitor(s) and optionally an additional anticancer agent wherein the G-CSF inhibitor(s) and optional additional anticancer agent are administered to the subject prior to or during radiation or other alternative therapy (e.g., hormonal therapy, proton therapy, cryosurgery, and/or high intensity focused ultrasound (HIFU), radiofrequency ablation, microwave ablation, transarterial therapies such as radioembolizationn with Y90 or bland embolization and chemoembolization (for liver cancer).

As described, in certain embodiments, the subject is also treated concomitantly by other anticancer agents, including chemotherapeutic agents such as agents which are DNA damaging agents, including such agents as paclitaxel and docetaxel, platinum-based antineoplastics (e.g. cisplatin, carboplatin, oxaliplatin, satraplatin, picoplatin, Nedaplatin, Triplatin, and Lipoplatin). In certain additional embodiments, further treatment of a cancer using hormonal therapy, proton therapy, cryosurgery, and/or high intensity focused ultrasound (HIFU), radiofrequency ablation, microwave ablation, transarterial therapies such as radioembolizationn with Y90 or bland embolization and chemoembolization (for liver cancer) is used, depending upon clinical assessments and treatment goals.

In certain embodiments, the subject suffers from a treatment-resistant (drug resistant) cancer, including a metastatic and/or recurrent cancer, such as, for example, a treatment resistant breast cancer; hormone and castration-resistant prostate cancer; metastatic melanoma; drug resistant childhood acute lymphoblastic leukemia (ALL); and chemotherapy and radiotherapy-resistant non-small cell lung cancer, glioblastomas, cervical cancer, esophageal cancer (EC) and colorectal cancers, among others.

In a further embodiment, the invention provides a method of treating a subject who suffers from cancer wherein the cancer has developed resistance to one or more cancer agents, the method comprising administering an effective amount of
(a) one or more G-CSF inhibitors; and optionally
(b) one or more additional anticancer agent to which the cells have not become resistant; and/or
(c) at least one anticancer agent (preferably, a chemotherapeutic agent such as a DNA damaging agent), wherein the method may be combined further with radiation therapy.

Pharmaceutical formulations that are useful in the treatment of a variety of cancers and inflammatory disorders are also provided. These formulations comprise (a) one or more elements or compounds selected from the group consisting of at least one G-CSF inhibitor as set forth herein; and optionally; -at least one additional anticancer agent and a pharmaceutically-acceptable excipient.

In certain embodiments, by combining G-CSF inhibitors with additional anticancer agents and further optionally, radiation therapy, the methods and formulations described herein prove particularly effective in treating a wide variety of cancers that have been previously been associated with high rates of remission, but poor long-term survival, especially when combined with a chemotherapy agent and optionally, radiation therapy.

These and other aspects of the invention are described further in the Detailed Description of the Invention.

### BRIEF DESCRIPTION OF THE FIGURES

FIG. 1: G-CSF is highly expressed in human tumors and by GI epithelial cells. G-CSF mRNA levels are increased in A) gastric and B) colon tumors compared to normal tissues as shown by tumor stage by quantitative real time PCR. C) Epithelial cells isolated from human tumors express EpCAM and D) and G-CSF in representative figures. E) In compiled data from multiple experiments, G-CSF expression is increased in epithelial cells isolated from gastric and colon tumors compared to epithelial cells from matched normal tissues when analyzed by flow cytometry. G-CSF is also produced in increased amounts by tumor-derived gastric and colon cancer fibroblasts/myofibroblasts as compared to matched normal tissue derived fibroblasts/myofibroblasts as shown in the media from such cultured cells by F) Luminex bead array. N=8 for E-F and the mean±standard error are shown as the results of multiple experiments. *p<0.05.

FIG. 2: G-CSFR is highly expressed in human tumors and by GI cancer epithelial cells. G-CSFR mRNA levels are increased in A) gastric and B) colon tumors compared to normal tissues as shown by tumor stage by quantitative real time PCR. C) G-CSFR is expressed on epithelial cells isolated from tumor samples and D) expression is increased in epithelial cells isolated from gastric and colon tumors compared to epithelial cells from matched normal tissues when analyzed by flow cytometry. G-CSFR is expressed on the surface of E) MKN and F) Caco-2 cells in representative histograms compared to solid peak isotype controls. N=8 for D and the mean±standard error are shown as the results of multiple experiments, *p<0.05.

FIG. 3: G-CSF induces proliferation of gastric and colon carcinoma cells. G-CSF treatment induces proliferation by CyQuant assay for DNA content of A) MKN-45 and AGS gastric carcinoma cells, B) Caco-2 and DLD 1 colon carcinoma cells. Proliferation was further verified by C) PCNA staining by flow cytometry. D) Tumor-derived GMF and CMF supernatants induced proliferation of MKN-45 and Caco-2, which was decreased upon adding anti-G-CSF neutralizing antibodies. The mean±standard error are shown as the results of multiple experiments, N=8*p<0.05 compared to untreated.

FIG. 4: G-CSF induces migration of gastric and colon carcinoma cells. Fluorescently labeled MKN-45 and Caco-2 cells were added to the top of Fluorblock™ plates with 8 μm pores with A) recombinant G-CSF and B) normal and tumor MF supernatants with G-CSF neutralization by monoclonal antibodies. Migration was assessed by mean fluourescence intensity. The mean±standard error are shown as the results of multiple experiments, N=8*p<0.05 compared to serum free media. Human tumor tissues from individuals that had cells migrate to lymph nodes have higher C) G-CSF in gastric cancer, D) G-CSF in colon cancer, E) G-CSFR in gastric cancer and F) G-CSFR in colon cancer.

FIG. 5: G-CSF increases a population of cells expressing stem-like markers in MKN-45 and Caco-2 cells. Aldefluor staining of MKN-45 cells gated on the CD44 positive population in representative dot plots as shown with A) DEAB Aldehyde dehydrogenase negative control inhibitor, B) untreated cells, C) G-CSF treated cells, and D) compiled data for MKN-45 and Caco-2 cells. For D, the mean±standard error are shown as the results of multiple experiments. N=8,*p<0.05.

FIG. 6: G-CSF induces ERK1/2 and RSK signaling. Recombinant G-CSF treatment induces A) ERK1/2 and B) RSK1 phosphorylation. The mean±standard error are shown as the results of multiple experiments. N=8,*p<0.05.

FIG. 7: Inhibition of ERK1/2 and RSK pathways reduces G-CSF induced proliferation and the increased population expressing stem-like markers. A) G-CSF induced cell proliferation is inhibited by ERK1/2 and RSK inhibitors as shown by fluorescent cell proliferation assay for DNA content (CyQuant®) and B) G-CSF induced expansion populations expressing CD44 and Aldehyde dehydrogenase is inhibited by ERK1/2 and RSK inhibitors. The mean±standard error are shown as the results of multiple experiments. N=8,*p<0.05.

FIG. 8 shows graphs A) human gastric and B) human colon cancers have significantly more G-CSF and G-CSFR expression as compared to matched normal tissue.

FIG. 9 shows graphs A) human gastric and B) human colon cancers have significantly more G-CSF and G-CSFR expression as compared to matched normal tissue and this is most pronounced in higher N stage tumors.

FIG. 10 is a photograph showing that Wild Type C57BL/6 mice treated with a carcinogen/inflammation model of colon cancer (AOM/DSS) develop multiple proximal tumors. With 8 mice, mean weight range for AOM/DSS of 20.96 gms (19.97-23.13), PBS 22.01 gms (21.19-23.89), and P=0.99 by X2, 7 of 8 mice developed a mean of 2.85 tumors/mouse (range 1-5) all of which were proximal to the mid colon.

FIG. 11 is a graph showing colon tumors/mouse in both G-CSFRKO mice and WT mice. This graph shows that G-CSFRKO mice had a trend towards fewer tumors per mouse than WT C57Bl/6 mice when treated with AOM/DSS tumor induction model.

FIG. 12: A and B are graphs showing that Wild Type C57BL/6 mice treated with a carcinogen/inflammation model of colon cancer (AOM/DSS) develop tumors (7/8) with significantly more G-CSF and G-CSFR than adjacent normal tissue.

FIG. 13 is a graph showing that Wild Type C57BL/6 mice treated with a carcinogen/inflammation model of colon cancer (AOM/DSS) have significantly more serum G-CSF than WT mice treated with PBS.

FIG. 14 is a graph showing that G-CSF treatment of gastric carcinoma cells significantly alters expression of 80 genes by 2 fold or greater.

FIG. 15 is a graph showing that (i) CRC tumor tissues secrete more G-CSF than normal colon tissue from the same patient and (ii) node positive primary tumor samples from human CRC secrete significantly more G-CSF than primary tumors from node negative samples.

FIG. 16 is a graph showing that Anti-G-CSF antibody decreases the tumor surface area in AOM/DSS model.

FIG. 17 is a graph showing that Anti-G-CSF antibody decreases the tumor number in AOM/DSS model.

### DETAILED DESCRIPTION OF THE INVENTION

The following terms are used throughout the specification to describe the present invention. Where a term is not given a specific definition herein, that term is to be given the same meaning as understood by those of ordinary skill in the art. The definitions given to the disease states or conditions which may be treated using one or more of the compounds according to the present invention are those which are generally known in the art.

It is noted that, as used in this specification and the appended claims, the singular forms “a,” “an,” and “the,” include plural referents unless expressly and unequivocally limited to one referent. Thus, for example, reference to “a compound” includes two or more different compounds. As used herein, the term “include” and its grammatical variants are intended to be non-limiting, such that recitation of items in a list is not to the exclusion of other like items that can be substituted or other items that can be added to the listed items.

The term “patient” or “subject” is used throughout the specification to describe an animal, preferably a human, to whom treatment, including prophylactic treatment, with the compositions according to the present invention is provided (a patient or subject in need). For treatment of those infections, conditions or disease states which are specific for a specific animal such as a human patient, the term patient refers to that specific animal. In many instances, diagnostic methods are applied to patients or subjects who are suspected of having cancer or who have cancer or a inflammatory disorder and the diagnostic method is used to assess the severity of the disease state or disorder.

The term “compound” is used herein to refer to any specific chemical compound disclosed herein and in particular, a G-CSF inhibitor, anticancer agent or other agent used in the present invention. Within its use in context, the term generally refers to a single small molecule as disclosed herein, but in certain instances may also refer to other forms of the compound, especially including polypeptides and antibodies, including poly- and monoclonal antibodies. The term compound includes active metabolites of compounds and/or pharmaceutically acceptable salts thereof.

The term “effective amount” is used throughout the specification to describe concentrations or amounts of formulations or other components which are used in amounts, within the context of their use, to produce an intended effect according to the present invention, for example to inhibit the effects of G-CSF, including a G-CSF receptor, to kill cells and/or damage DNA as a chemotherapy agent or by exposure to radiation or other alternative therapy as described herein. The formulations or component(s) may be used to produce a favorable change in a disease or condition treated, whether that change is a remission of effects of a disease state or condition, a favorable physiological result, a reversal or attenuation of a disease state or condition treated, the prevention or the reduction in the likelihood of a condition or disease-state occurring, depending upon the disease or condition treated. Where formulations are used in combination, each of the formulations is used in an effective amount, wherein an effective amount may include a synergistic amount. The amount of formulation used in the present invention may vary according to the nature of the formulation, the age and weight of the patient and numerous other factors which may influence the bioavailability and pharmacokinetics of the formulation, the amount of formulation which is administered to a patient generally ranges from less than about 0.001 mg/kg to about 50 mg/kg or more, about 0.1 mg/kg to about 7.5 mg/kg, about 0.5 mg/kg to about 25 mg/kg, about 0.1 to about 15 mg/kg, about 1 mg to about 10 mg/kg per day and otherwise described herein. The person of ordinary skill may easily recognize variations in dosage schedules or amounts to be made during the course of therapy.

G-CSF inhibitors include any compound which is capable of preventing the interaction of G-CSF with its receptor and includes those compounds which bind to G-CSF such as anti-human G-CSF monoclonal antibody clone 4A4RC (EBiosciences) and anti-mouse G-CSF antibody (R&D Systems), small molecules which inhibit the binding of G-CSF to the G-CSF receptor and compounds which bind to the G-CSF receptor and prevent binding at the receptor site (anti-human G-CSF receptor monoclonal antibody and anti-mouse G-CSF receptor antibody, including humanized antibody CSL 324, available from CSL Behring company—which is an antibody against human G-CSFR). All of these compounds, among others, may be used to inhibit G-CSF and consequently, treat cancer, including metastatic and recurrent cancer, through the inhibition of the growth, elaboration and metastasis and reduce the likelihood of metastasis and/or recurrence of a cancer which has gone into remission. One or more of these compounds may be used alone or in combination with at least one additional anticancer agent, and optionally radiation and other anticancer therapy in the treatment of cancer.

The term “prophylactic” is used to describe the use of a formulation described herein which reduces the likelihood of an occurrence of a condition or disease state in a patient or subject. The term “reducing the likelihood” refers to the fact that in a given population of patients, the present invention may be used to reduce the likelihood of an occurrence, recurrence or metastasis of disease in one or more patients within that population of all patients, rather than prevent, in all patients, the occurrence, recurrence or metastasis of a disease state.

The term “pharmaceutically acceptable” refers to a salt form or other derivative (such as an active metabolite or prodrug form) of the present compounds or a carrier, additive or excipient which is not unacceptably toxic to the subject to which it is administered.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc. Treatment, as used herein, encompasses both prophylactic and therapeutic treatment.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Cancers generally show partial or complete lack of structural organization and functional coordination with the normal tissue and most invade surrounding tissues, metastasize to several sites, and are likely to recur after attempted removal (recurrent cancer) and to cause the death of the patient unless adequately treated.

As used herein, the term cancer is used to describe all cancerous disease states applicable to treatment according to the present invention and embraces or encompasses the pathological process associated with all virtually all epithelial cancers, including carcinomas, malignant hematogenous, ascitic and solid tumors. Examples of cancers which may be treated using methods according to the present invention include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias (various); benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas. See, for example, The Merck Manual of Diagnosis and Therapy, 17.sup.th ed. (Whitehouse Station, N.J.: Merck Research Laboratories, 1999) 973-74, 976, 986, 988, 991).

In addition to the treatment of ectopic cancers as described above, the present invention also may be used preferably to treat eutopic cancers such as choriocarcinoma, testicular choriocarcinoma, non-seminomatous germ cell testicular cancer, placental cancer (trophoblastic tumor)and embryonal cancer, among others.

The term “neoplasia” refers to the uncontrolled and progressive multiplication of tumor cells, under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive. Thus, neoplasia includes “cancer”, which herein refers to a proliferation of tumor cells having the unique trait of loss of normal controls, resulting in unregulated growth, lack of differentiation, local tissue invasion, and/or metastasis.

As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of anaplasia, or loss of differentiation and orientation of cells, and have the properties of invasion and metastasis. Examples of (many of which are identified above) include neoplasms or neoplasias from which the target cell of the present invention may be derived include, without limitation, carcinomas (e.g., squamous-cell carcinomas, basal cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991.

The term “anticancer agent” or “additional anticancer agent” shall mean chemotherapeutic agents including such as an agent selected from the group consisting of microtubule-stabilizing agents, microtubule-disruptor agents, alkylating agents, antimetabolites, epidophyllotoxins, antineoplastic enzymes, topoisomerase inhibitors, inhibitors of cell cycle progression, and platinum coordination complexes. These may be selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bc1-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111 , 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N -[4-[2-(2-amino-4,7-dihydro-4-oxo-1 H -pyrrolo[2,3-d ]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D- Ser(Bu t) 6,Azgly 10] (pyro-Glu-His-Trp-Ser-Tyr-D-Ser(Bu t)-Leu-Arg-Pro- Azgly-NH ₂ acetate [C₅₉H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytam oxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, among others.

The term “DNA damaging agent” refers to a chemotherapeutic agent which may be used as an additional anticancer agent in the methods of the present invention, which specifically damages DNA of a cancer cell either directly or indirectly in its actions. Many chemotherapy agents are considered DNA damaging agents. Preferred agents include alkylating agents, including nitrogen mustards: such as mechlorethamine (nitrogen mustard), chlorambucil, cyclophosphamide (Cytoxan®), ifosfamide, and melphalan; Nitrosoureas, including streptozocin, carmustine (BCNU), and lomustine; Alkyl sulfonates, including busulfan; Triazines, including dacarbazine (DTIC) and temozolomide (Temodar®); Ethylenimines, including thiotepa and altretamine (hexamethylmelamine); Platinum drugs, including cisplatin, carboplatin and oxalaplatin; Antimetabolites including fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, Thioguanine; Anti-tumor antibiotics including Anthracyclines, such as Daunorubicin, Doxorubicin (Adriamycin®), Epirubicin, Idarubicin and non-anthracycline antibioitics Actinomycin-D , Bleomycin and Mitomycin-C; Topoisomerase inhibitors including topotecan and irinotecan (CPT-11), etoposide (VP-16), teniposide and Mitoxantrone; Mitotic inhibitors, including Taxanes: paclitaxel (Taxol®) and docetaxel (Taxotere®); Epothilones, including ixabepilone (Ixempra®); Vinca alkaloids, including vinblastine (Velban®), vincristine (Oncovin®), and vinorelbine (Navelbine®), Estramustine (Emcyt®); and Targeted therapies including imatinib (Gleevec®), gefitinib (Iressa®), sunitinib (Sutent®) and bortezomib (Velcade®), among others.

The terms “radiotherapy” and “radiation therapy” are used interchangeably and describe therapy for cancer, especially including prostate cancer, which may be used in conjunction with certain G-CSF inhibitor compounds in combination with other agents, including those having radiation sensitization activity. Radiation therapy uses high doses of radiation, such as X-rays or other energy sources such as radioisotopes (gamma, beta or alpha emitters), to destroy cancer cells. The radiation damages the genetic material of the cells so that they cannot grow. Although radiation damages normal cells as well as cancer cells, the normal cells can repair themselves and function, while the cancer cells cannot.

Radiation therapy may be used in combination with the presently claimed compounds, alone or in combination with additional anticancer compounds as otherwise disclosed herein, depending on the cancer to be treated, and consequently, the cancer cells' ability to repair damage done by the radiation, thus potentiating radiation therapy. Radiation therapy is most effective in treating cancers that have not spread (metastasized). But it also may be used if the cancer has spread to nearby tissue. Radiation is sometimes used after surgery to destroy any remaining cancer cells and to relieve pain from metastatic cancer.

Radiation is delivered in one of two ways: External-beam radiation therapy and brachytherapy. External-beam radiation therapy uses a large machine to aim a beam of radiation at the tumor. After the area of cancer is identified, an ink tattoo no bigger than a pencil tip is placed on the skin of the subject so that the radiation beam can be aimed at the same spot for each treatment. This helps focus the beam on the cancer to protect nearby healthy tissue from the radiation. External radiation treatments usually are done 5 days a week for 4 to 8 weeks or more. If cancer has spread, shorter periods of treatment may be given to specific areas to relieve pain.

There are basically three types of external radiation therapy: conformal radiotherapy (3D-CRT), intensity-modulation radiation therapy (IMRT) and proton therapy. Conformal radiotherapy uses a three-dimensional planning system to target a strong dose of radiation to the cancer. This helps to protect healthy tissue from radiation. Intensity-modulated radiation therapy uses a carefully adjusted amount of radiation. This protects healthy tissues more than conformal radiotherapy does. Proton therapy uses a different type of energy (protons) than X-rays. This approach allows a higher amount of specifically directed radiation, which protects nearby healthy tissues the most. Sometimes proton therapy is combined with X-ray therapy.

Brachytherapy, or internal radiation therapy, uses dozens of tiny seeds that contain radioactive material. It may be used preferably to treat early-stage prostate and other cancer which is localized. Needles are used to insert the seeds through the skin into tissue, most often the prostate. The surgeon uses ultrasound to locate the tissue and guide the needles. As the needles are pulled out, the seeds are left in place. The seeds release radiation for weeks or months, after which they are no longer radioactive. The radiation in the seeds can't be aimed as accurately as external beams, but they are less likely to damage normal tissue. After the seeds have lost their radioactivity, they become harmless and can stay in place.

Radiation therapy may combine brachytherapy with low-dose external radiation. In other cases, treatment combines surgery with external radiation. In the present invention, compounds which are otherwise claimed may be used as radiation sensitizers to enhance or potentiate the effect of radiation by inhibiting the ability of the cancer tissue to repair the damage done by the radiation therapy.

Other alternative therapies which can be used in combination with G-CSF inhibitors and optionally radiation therapy, include for example hormonal therapy, proton therapy, cryosurgery, and/or high intensity focused ultrasound (HIFU), radiofrequency ablation, microwave ablation, transarterial therapies such as radioembolization with Y90 or bland embolization and chemoembolization (for liver cancer).

Formulations of the invention may include a pharmaceutically acceptable diluent, carrier, solubilizer, emulsifier, preservative and/or adjuvant. Acceptable formulation materials preferably are nontoxic to recipients at the dosages and concentrations employed. The pharmaceutical formulations may contain materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, polyethylene glycol (PEG), sorbitan esters, polysorbates such as polysorbate 20 and polysorbate 80, Triton, trimethamine, lecithin, cholesterol, or tyloxapal); stability enhancing agents (such as sucrose or sorbitol); tonicity enhancing agents (such as alkali metal halides, preferably sodium or potassium chloride, mannitol, or sorbitol); delivery vehicles; diluents; excipients and/or pharmaceutical adjuvants. See, for example, REMINGTON'S PHARMACEUTICAL SCIENCES, 18.sup.th Edition, (A. R. Gennaro, ed.), 1990, Mack Publishing Company.

Optimal pharmaceutical formulations can be determined by one skilled in the art depending upon, for example, the intended route of administration, delivery format and desired dosage. See, for example, REMINGTON'S PHARMACEUTICAL SCIENCES, Id. Such formulations may influence the physical state, stability, rate of in vivo release and rate of in vivo clearance of the antibodies of the invention.

Primary vehicles or carriers in a pharmaceutical formulation can include, but are not limited to, water for injection, physiological saline solution or artificial cerebrospinal fluid, possibly supplemented with other materials common in compositions for parenteral administration. Neutral buffered saline or saline mixed with serum albumin are further exemplary vehicles. Pharmaceutical formulations can comprise Tris buffer of about pH 7.0-8.5, or acetate buffer of about pH 4.0-5.5, which may further include sorbitol or a suitable substitute. Pharmaceutical formulations of the invention may be prepared for storage by mixing the selected composition having the desired degree of purity with optional formulation agents (REMINGTON'S PHARMACEUTICAL SCIENCES, Id.) in the form of a lyophilized cake or an aqueous solution. Further, the formulations may be formulated as a lyophilizate using appropriate excipients such as sucrose.

Formulation components are present in concentrations that are acceptable to the site of administration. Buffers are advantageously used to maintain the composition at physiological pH or at a slightly lower pH, typically within a pH range of from about 5 to about 8.

The phaimaceutical formulations of the invention can be delivered parenterally. When parenteral administration is contemplated, the therapeutic formulations for use in this invention may be in the form of a pyrogen-free, parenterally acceptable aqueous solution. Preparation involves the formulation of the desired immunomicelle, which may provide controlled or sustained release of the product which may then be delivered via a depot injection. Formulation with hyaluronic acid has the effect of promoting sustained duration in the circulation.

Formulations may be formulated for inhalation. In these embodiments, a stealth immunomicelle formulation is formulated as a dry powder for inhalation, or inhalation solutions may also be formulated with a propellant for aerosol delivery, such as by nebulization. Pulmonary administration is further described in PCT Application No. PCT/US94/001875, which describes pulmonary delivery of chemically modified proteins and is incorporated by reference.

Formulations of the invention can be delivered through the digestive tract, such as orally. The preparation of such pharmaceutically acceptable compositions is within the skill of the art. Formulations disclosed herein that are administered in this fashion may be formulated with or without those carriers customarily used in the compounding of solid dosage forms such as tablets and capsules. A capsule may be designed to release the active portion of the formulation at the point in the gastrointestinal tract when bioavailability is maximized and pre-systemic degradation is minimized Additional agents can be included to facilitate absorption. Diluents, flavorings, low melting point waxes, vegetable oils, lubricants, suspending agents, tablet disintegrating agents, and binders may also be employed.

A formulation may involve an effective quantity of a micropoarticle containing formulation as disclosed herein in a mixture with non-toxic excipients that are suitable for the manufacture of tablets. By dissolving the tablets in sterile water, or another appropriate vehicle, solutions may be prepared in unit-dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

The pharmaceutical composition to be used for in vivo administration typically is sterile. In certain embodiments, this may be accomplished by filtration through sterile filtration membranes. In certain embodiments, where the composition is lyophilized, sterilization using this method may be conducted either prior to or following lyophilization and reconstitution. In certain embodiments, the composition for parenteral administration may be stored in lyophilized form or in a solution. In certain embodiments, parenteral compositions generally are placed into a container having a sterile access port, for example, an intravenous solution bag or vial having a stopper pierceable by a hypodermic injection needle.

Once the formulation of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

Administration routes for formulations of the invention include orally, through injection by intravenous, intraperitoneal, intracerebral (intra-parenchymal), intracerebroventricular, intramuscular, intra-ocular, intraarterial, intraportal, intrathecal or intralesional routes; by sustained release systems or by implantation devices, transdermally or topically among other routes of administration, including bucally or via suppository. The pharmaceutical formulations may be administered by bolus injection or continuously by infusion, or by implantation device. The pharmaceutical formulations also can be administered locally via implantation of a membrane, sponge or another appropriate material onto which the desired molecule has been absorbed or encapsulated. Where an implantation device is used, the device may be implanted into any suitable tissue or organ, and delivery of the desired molecule may be via diffusion, timed-release bolus, or continuous administration.

### G-CSF Inhibitor and G-CSFR Inhibitor

Preferred methods of treatment and pharmaceutical formulations include the following.

In one embodiment, the invention provides a method of treating a subject who suffers from a cancer (any cancer as otherwise disclosed herein), preferably a cancer selected from the group consisting of breast cancer, ovarian cancer, lung cancer, colorectal cancer, glioblastoma multiform (GBM), melanoma, glioma, esophageal cancer, gastric cancer, heptacellular cancer, gallbladder cancer, cholangiocarcinoma cancer, prostate cancer, cervical cancer, uterine cancer, sarcomas, renal cancer, bladder cancer and pancreatic cancer,the method comprising co-administering to the subject a pharmaceutically-effective amount of:
(a) one or more elements or compounds which is a G-CSF inhibitor;
(b) optionally, one or more additional anticancer agents, including a chemotherapy agent (preferably, at least one DNA-damaging agent); and
(c) optionally, employing radiation or other alternative therapy.

In a preferred embodiment, the subject is treated concomitantly by radiotherapy and the one or more G-CSF compounds is combined with an agent are administered to the subject as a radiosensitizer prior to or during radiotherapy, optionally in combination with at least one additional anticancer agent as otherwise disclosed herein.

In certain embodiments, the subject suffers from a treatment-resistant cancer selected from the group consisting of breast cancer in which BRCAl-deficient cells exhibit decreased sensitivity to PARP inhibitors; ovarian cancer which is resistant to platinum-containing anti-neoplastic drugs; hormone and castration-resistant prostate cancer; metastatic melanoma; drug resistant childhood acute lymphoblastic leukemia (ALL); and chemotherapy and radiotherapy-resistant glioblastomas, cervical cancer, esophageal cancer (EC), breast cancers and non-small cell lung cancer.

Preferably, the G-CSF agent is selected from the group consisting of anti-human G-CSF monoclonal antibody, such as clone 4A4RC (EBiosciences), small molecules which inhibit the binding of G-CSF to the G-CSF receptor and compounds which bind to the G-CSF receptor and prevent binding at the receptor site (anti-human G-CSF receptor monoclonal antibody.

In further embodiments, the G-CSF inhibition may be combined with at least one additional anticancer agent including, for example, at least one additional PARP inhibitor selected from the group consisting of arsenic trioxide (ATO), NU1025; 3-aminobenzamide; 4-amino-1,8-naphthalimide; 1,5-isoquinolinediol; 6(5H)-phenanthriddinone; 1,3,4,5,-tetrahydrobenzo(c) (1,6)-and (c)(1,7)-naphthyridin-6 ones; adenosine substituted 2,3-dihydro-1H-isoindol-1-ones; AG14361; AG014699; 2-(4-chlorophenyl)-5-quinoxalinecarboxamide; 5-chloro-2-[3-(4-phenyl-3,6-dihydro-1(2H)-pyridinyl)propyl]-4(3H)-quinazolinone; isoindolinone derivative INO-1001; 4-hydroxyquinazoline; 2-[3-[4-(4-chlorophenyl) 1-piperazinyl]propyl]-4-3(4)-quinazolinone; 1,5-dihydroxyisoquinoline (DHIQ); 3,4-dihydro-5[4-(1-piperidinyl)(butoxy)-1(2H)-isoquinolone; CEP-6800; GB-15427; PJ34; DPQ; BS-201; AZD2281 (Olaparib); BS401; CHP101; CHP102; INH2BP; BSI201; BSI401; TIQ-A; an imidazobenzodiazepine; 8-hydroxy-2-methylquinazolinone (NU1025), CEP 9722, MK 4827, LT-673; 3-aminobenzamide; Olaparib (AZD2281; ABT-888 (Veliparib); BSI-201 (Iniparib); Rucaparib (AG-014699); INO-1001; A-966492; PJ-34; and the PARP1 inhibitors described in U.S. patent application Ser. No. 12/576,410.

The subject treated in the embodiment of the preceding paragraph may suffer from one or more cancers, including a cancer selected from the group consisting of breast cancer, ovarian cancer, non-small-cell lung cancer, prostate cancer, colorectal cancer, liver cancer and gastrointestinal cancer.

In certain embodiments, a subject treated by the methods of treatment of the invention suffers from one or more cancers selected from the group consisting of relapsed or refractory T-cell prolymphocytic leukemia (T-PLL), chronic lymphocytic leukemia (CLL), locally advanced or metastatic colorectal carcinoma (CRC), persistent or recurrent endometrial carcinoma, locally advanced or metastatic triple negative or highly proliferative estrogen receptor positive (ER+) breast cancer and partially platinum-sensitive epithelial ovarian cancer.

Another preferred embodiment provides a method of treating a subject who suffers from a solid tumor, the method comprising co-administering to the subject a pharmaceutically-effective amount of:
(a) one or more G-CSF inhibitors; and
(b) optionally, at least one additional anticancer agent. This method may also be used in combination with radiation or other alternative therapy.

These and other aspects of the invention are illustrated further in the following non-limiting Examples.

### EXAMPLES

Background: Granulocyte colony-stimulating factor (G-CSF) is a pro-inflammatory cytokine that stimulates myeloid stem cell maturation, proliferation, and migration into circulation. Despite being a known growth factor, the impact of G-CSF on solid tumors has not been well examined. G-CSF receptor (G-CSFR) is expressed by some tumors, and thus the aim of this study was to examine the expression and impact of G-CSF and G-CSFR on gastrointestinal tumors.

### Methods:

Tissue and Cell Culture: All human samples were collected under IRB approved human protocols at the University of New Mexico Health Sciences Center and the Legacy Research Institute Tumor Bank where patients gave written consent. Epithelial cells and primary cultures of MF from matched normal and cancer tissues were attained by a series of EDTA treatments and enzymatic cell dissociation using the GentleMACS system (Miltenyi Biotech, Bergisch Gladbach, Germany) and cultured as previously described (Saada et al., 2006). Epithelial cells were used for the flow cytometry. MFs were isolated according to the protocol routinely used in our laboratory (pinchuk et al., 2013; Saada et al., 2006 ). The purity of isolated CD90⁺and α-smooth smooth actin⁺(α-SMA) MFs (98-99%) was confirmed by flow cytometry, as previously described (19). Cells were cultured in complete Modified Eagle Medium (MEM) with 10% FBS. MKN-45 cells were obtained from Dr. Yoshio Yamaoka at Baylor College of Medicine and AGS cells were obtained from American Type Tissue Culture (ATCC, Manassas, Va.) and maintained in RPMI with 10% FBS and 2 mM L-glutamine. Caco-2 and DLD1 cells were obtained from ATCC and maintained in DMEM with 10% FBS and 2 mM L-glutamine.

Real-Time PCR: RNA was isolated using trizol (Life Technologies, Grand Island, N.Y.) according to the manufacturer's instructions. RNA concentrations were measured using a Nanodrop instrument (Thermo Scientific, Wilmington, Del.). Real-time PCR was performed according to Applied Biosystems′ two-step protocol. The RT reaction mixture includes random 2.5 μM hexamers, 500 μM dNTPs, 0.4 U/λL of the RNase inhibitors, 5.5 mM MgCl₂, MultiScribe Reverse Transcriptase (3.125 U/μL) and its buffer, and 1 μg of cellular RNA. The RT step was performed according to the following protocol: 10 min at 25° C., 60 min at 37° C., 5 min at 95° C. Obtained cDNA samples were stored at −80° C. and used for the PCR reaction step. The PCR reaction mix was prepared using the Assays-on-Demand™ gene expression assay mix (Applied Biosystems) for human 18S, G-CSF, and G-CSFR (a 20× mix of unlabeled PCR primers and TaqMan® MGB probe, FAM dye-labeled) and 2 μL of cDNA were added to the PCR reaction mix. The reaction was carried out according to the following protocol: 2 min at 50° C., 10 min at 95° C. (1 cycle), and 15 sec at 95° C. and one min at 60° C. (45 cycles) on Applied Biosystem's StepOnePlus instrument. The endpoint used in real-time PCR quantification, CT, was defined as the PCR cycle number that crossed the signal threshold. Quantification of cytokine gene expression was performed using the comparative CT method (Sequence Detector User Bulletin 2; Applied Biosystems) and reported as the fold difference relative to the human housekeeping gene, 18S mRNA.

Flow Cytometry: Single-and multi-color immunostaining was performed according to standard surface and intracellular FACS staining Biolegend protocols (Biolegend, San Diego, Calif.). Anti-Ep-cam Alexa-Fluor 488 (Biolegend 9c4) was used for identification of epithelial cells. The purity of MF was analyzed by staining for CD90-PE (Biolegend Thyl) and α-smooth actin-FITC (R&D Systems 1C14206) for flow cytometry compared to isotype controls. Anti-G-CSFR-PE (clone 1 mm 741 Biolegend) and G-CSF-FITC (clone 85FSCSF eBioscience) were used for the analysis of G-CSFR surface expression and intracellular G-CSF after 4 hour exposure to Brefeldin A compared to isotype controls. Anti-proliferating cell nuclear antigen (PCNA) (clone PCNA01, Biolegend) was used as a proliferation marker. Cancer stem-like populations in carcinoma cell lines were examined by staining with Aldefluor (Stem Cell Technologies, Vancouver, BC) and anti-CD44-APC (Biolegend clone BJ18) using the manufacturer's Aldefluor staining protocol compared to isotype control. All samples were analyzed on a Guava easyCyte 8HT flow cytometer (Millipore, Bellerica, Md.), and analyzed using FCS Express software (DeNovo Software, Los Angeles, Calif.).

Luminex Arrays: G-CSF levels were measured in cell culture supernatants by singleplex fluorescent bead based array (Life Technologies) according to manufacturer's instructions. ERK1/2 and RSK phosphorylation were measured by Milliplex MAPmates phospho-ERK1/2 and RSK arrays (EMD Millipore, Billerica Mass.) according to the manufacturer protocol. All samples were analyzed on a Luminex 200 machine.

Proliferation: MKN-45, AGS, Caco-2 and DLD1 cells (2500 per well) were added to wells of a 96 well plate and incubated with 5-25 ng/ml of recombinant G-CSF (eBioscience) or conditioned media from MF for 48 hours. Conditioned media included a mixture of 50% media from cultured myofibroblasts and 50% fresh media for nutrients. Media from MF was taken when cells were 80% or greater confluent before passage (from approximately 1×10⁶ cells). Proliferation was measured using CyQuant dye for DNA content (Life Technologies). Other samples were incubated with 10 μM ERK activation inhibitor peptide 1 or RSK inhibitor SL0101 (EMD Millipore) for 30 min before addition of recombinant G-CSF. Samples were read on a Tecan fluorescent plate reader (Mannedorf, Switzerland).

Migration: MKN-45 and Caco-2 cells were stained with carboxyfluorein succinimidyl ester (CFSE) (Life Technologies) and added to the top of Fluoroblok 96 well plates with 8.0 μM pores. Serum free media, media with 10% FBS, recombinant G-CSF, or MF supernatants were added to the bottom of the wells. G-CSF neutralizing antibodies (1 μg/ml) were added to some cultures. The fluorescence of migrated cells was measured after 4 hours of incubation at 37° C. on a Tecan plate reader.

Statistical Analysis: Results were expressed as the mean±SE of data obtained from at least three independent experiments done with triplicate sets in each experiment. Differences between means were evaluated by ANOVA using Student's t-test for multiple comparisons in GraphPad Prism 5. Values ofp<0.05 were considered statistically significant.

### Results:

### Human Gastric and Colon Epithelial Cells and Tumor Derived Fibroblasts Produce G-CSF:

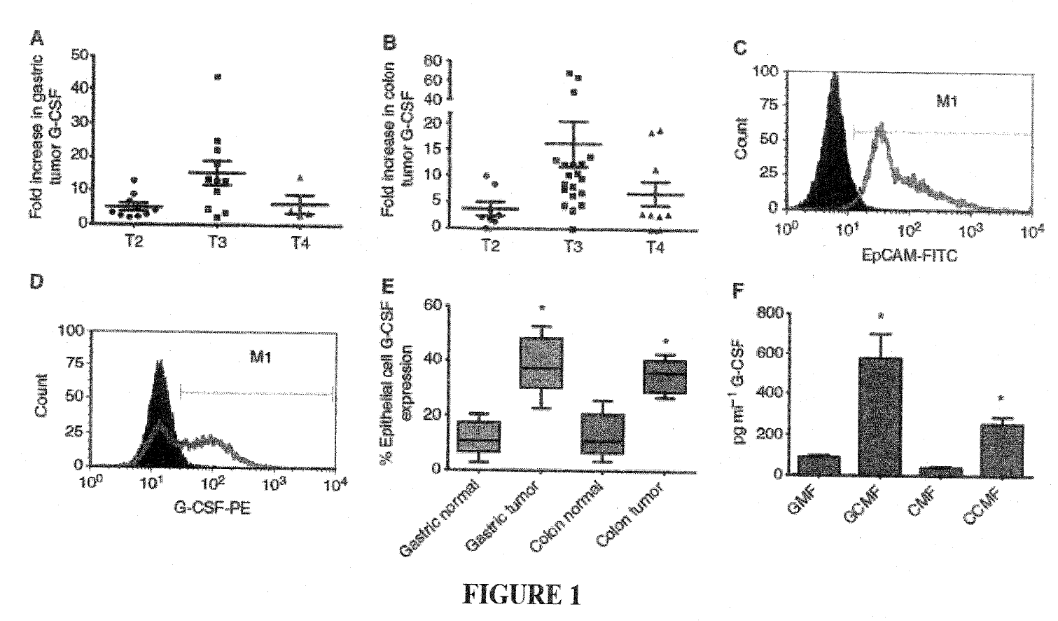

The pro-inflammatory nature of G-CSF and its ability to induce proliferation pathways led us to examine expression in human tumors. The mRNA levels were first examined in human gastric and colon tumors compared to matched normal tissues from the same individual. Twenty-six gastric tumors were examined with 25 of the 26 exhibiting a greater than 2-fold increase in G-CSF mRNA levels (FIG. 1A). These data were further examined with regards to tumor stage. Tumor stage 3 (T3) samples were found to have significantly higher G-CSF expression than T2 and T4. The mean fold increase in G-CSF mRNA levels over matched normal tissues for T2 was 5.13, T3 was 15.25, and T4 was 6.22. Similar results were seen with colon tumors (FIG. 1B) where T3 had significantly higher G-CSF mRNA levels with the mean for T2 at 3.65, T3 at 16.14, and T4 at 7.16-fold increase over normal tissues. Demographic and grade data were available for 5 of the gastric cancer patients and 14 of the colon cancer patients. There were 3 males and 2 females all with high grade tumors in the gastric cancer group. The colon cancer group had 8 males and 6 females, with 3 patients having high grade tumors, 8 having intermediate grade tumors, and 3 having low grade tumors. There were no significant differences between the fold increases in G-CSF and G-CSFR tumor expression when stratified by sex or tumor grade. In order to further examine G-CSF production, epithelial cells were isolated from tissues as previously described (Saada et al., 2006). After isolation, >95% of cells were epithelial cells as evidenced by staining with Ep-Cam for flow cytometry (FIG. 1C). Isolated epithelial cells from T3 samples were stained for G-CSF intracellularly (FIG. 1D) and data from both gastric and colon tumors reveal a significant increase in G-CSF expression (up to 30% increase) was seen compared to matched normal tissues from the same individuals (FIG. 1E). In addition to epithelial cells isolated from tumors, tumor-associated fibroblasts/myofibroblasts were also examined. These cells are known to produce inflammatory cytokines and growth factors and our group routinely cultures these cells from human tissues (Pinchuk et al., 2013; Saada et al., 2006). Thus, we found cultured tumor-derived fibroblasts/myofibroblasts (GMF for gastric and CMF for colon) to produce significant amounts of G-CSF (FIG. 1F). By Luminex fluorescent singleplex bead array, GMF were found to produce up to 600 pg/ml of G-CSF in supernatants compared to 50 pg/ml for normal tissue-derived cells, and CMF were found to produce up to 300 pg/ml of G-CSF in supernatants while normal colon tissue-derived cells were found to produce 25 mg/ml on average. These data suggest that both gastric and colon tumors produce G-CSF, by both carcinoma cells and tumor-associated fibroblasts/myofibroblasts.

### Human Gastric and Colon Tumors Express G-CSFR:

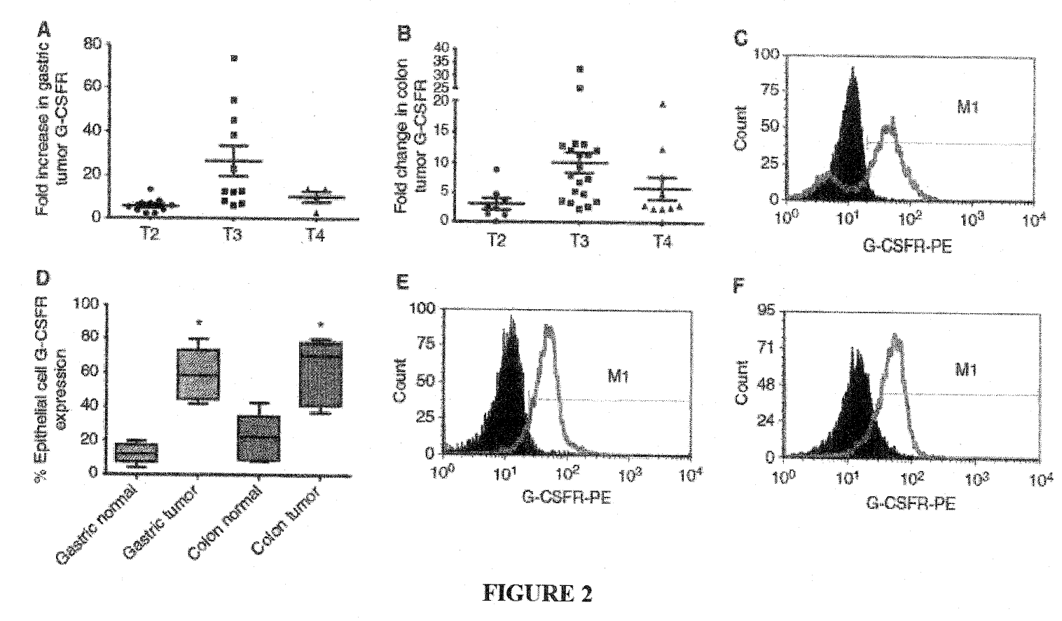

Since G-CSF is known to induce proliferation of some cell types through induction of G-CSFR signaling, the expression of G-CSFR was examined in human gastric and colon tumors. The G-CSFR mRNA level in tumor samples was compared to that in matched normal gastric or colon tissues from the same patient by quantitative real-time RT-PCR (qRT-PCR). Twenty-six gastric tumors were examined with 25 exhibiting a greater than 2-fold increase in G-CSFR mRNA levels. The fold increase in mRNA levels ranged from 2.3 to 73.9, with 15 samples having over a 5-fold increase (FIG. 2A). When samples were examined by tumor stage, stage 3 (T3) had a significantly higher mean than T2 and T4. The mean fold increase in G-CSFR levels in tumors over normal tissues for T2 was 7.96, T3 was 26.80, and T4 was 10.68. Thirty-nine colorectal cancer samples were also examined with 35 exhibiting a greater than 2-fold increase in G-CSFR mRNA levels over the matched normal colon tissue. The increase ranged from 2.1 to 32.7-fold with 13 samples having over a 5-fold increase (FIG. 2B). When samples were examined by tumor stage, T3 had a significantly higher mean similar to the gastric tumor samples with T2 having a mean fold increase of 3.57, T3 was 10.06, and T4 was 5.8 in tumors over matched normal tissues. To assure the over-expression was on the epithelial cells as opposed to local infiltration of immune cells, epithelial cells were isolated from tissues as previously described (Saada et al., 2006). As described above, >95% of isolated cells were epithelial cells as evidenced by staining with Ep-Cam for flow cytometry (not shown). Upon staining epithelial cells for G-CSFR surface expression for flow cytometry, approximately 60% of tumor-derived epithelial cells were found to express G-CSFR compared to 10% of normal gastric epithelial cells and 20% of normal colon epithelial cells (FIG. 2C-D). Since functional assays require the use of cell lines, G-CSFR expression was also examined on the surface MKN-45 gastric carcinoma cells and Caco-2 colorectal carcinoma cells. FIGS. 2E and F demonstrate the presence of G-CSFR on both MKN-45 and Caco-2 cell surfaces when analyzed by flow cytometry. G-CSFR expression was also seen with AGS gastric carcinoma cells and DLD1 colon carcinoma cells (not shown). In order to compare expression of G-CSFR on human samples to cell lines, the mean fluorescence intensities (MFI) were compared in flow cytometry data from FIGS. 2C-E, and MFI of cell lines was within 12% of the median for cancer cells, indicating that these are relevant cell line models with similar expression levels as the primary tumor samples. We also examined the cell lines for production of G-CSF and found low basal levels of production which was significantly increased when they were exposed to lipopolysaccharide (data not shown). The increased expression of the receptor in gastric and colon tumors and the expression on carcinoma cell lines suggest a need to examine the effects of G-CSF on GI tumor cells.

G-CSF induces gastrointestinal carcinoma cell proliferation:

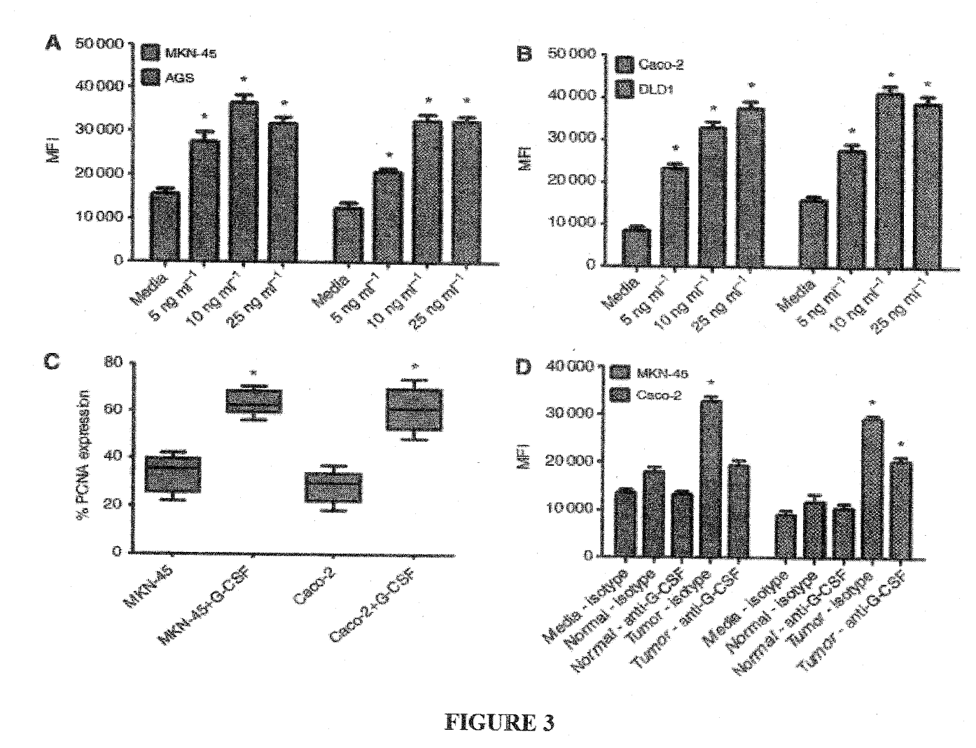

Since G-CSF has been shown to induce the proliferation of myeloid cells as well as head and neck squamous cancer cells (Gutschalk et al., 2006), we examined the impact of G-CSF on gastric and colon carcinoma cells. MKN-45 and AGS gastric carcinoma cells and Caco-2 and DLD1 colon carcinoma cells were incubated with increasing doses of recombinant G-CSF (5-25 ng/ml), which were chosen based on reports of serum concentrations in patients (Morstyn et al., 1989). After 4 days (approximately 2 doubling times), proliferation was measured using the CyQuant fluorescent proliferation assay for DNA content. G-CSF treated cells were compared to untreated control cells. Both gastric and colon carcinoma cells exhibited a dose dependent increase in proliferation compared to untreated cells, with up to a 2.5-fold increase in MKN-45 and AGS proliferation and up to 4.5-fold increase in Caco-2 and DLD1 colon carcinoma cell proliferation (FIGS. 3A and B). To further confirm G-CSF induction of cell proliferation, G-CSF treated cells were stained intracellularly for proliferating cell nuclear antigen (PCNA). A greater than 2-fold increase in PCNA staining was seen on flow cytometry for G-CSF treated cells as compared to untreated cells for both cell types (FIG. 3C). Since tumor-derived fibroblasts/myofibroblasts were found to produce high levels of G-CSF, supernatants from these cells isolated from human tissues were incubated with MKN-45 and Caco-2 cells in proliferation assays (FIG. 3D). Proliferation of carcinoma cells was found to increase significantly upon incubation with conditioned media from cancer derived fibroblasts/myofibroblasts, but was decreased upon addition of G-CSF neutralizing antibodies. These data suggest possible autocrine and paracrine activity of G-CSF on GI carcinoma cells.

### G-CSF Induces Gastrointestinal Carcinoma Cell Migration:

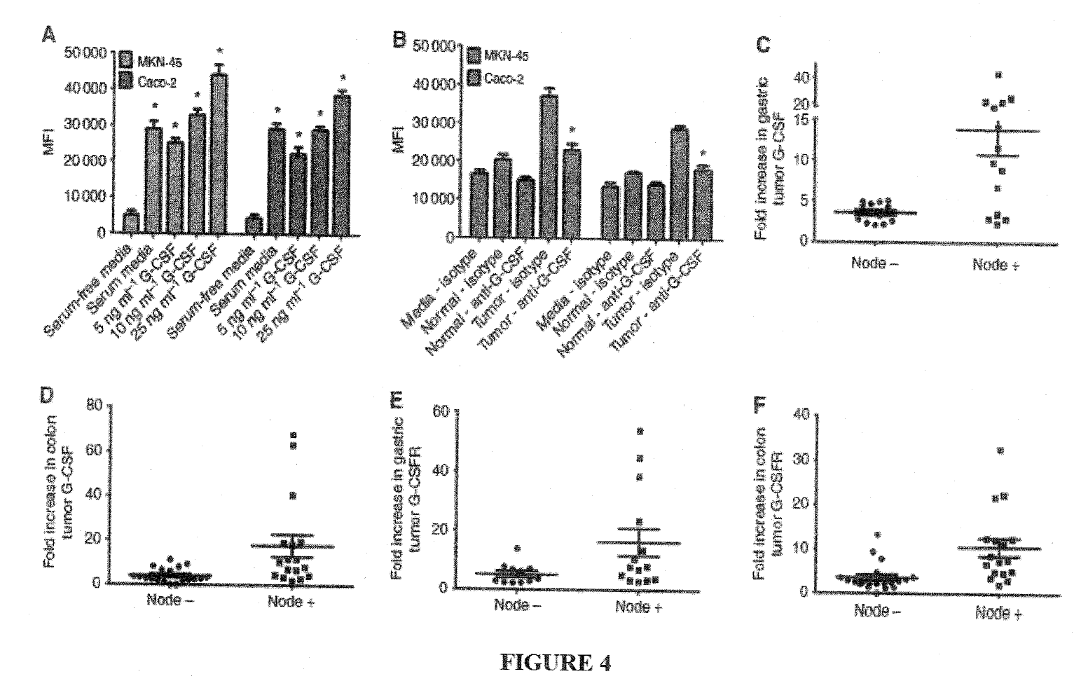

Given the ability of G-CSF to mobilize bone marrow derived hematopoietic cells during systemic administration (Held and Gundert-Remy, 2010), we assessed whether it could increase the migratory ability, and therefore the malignant potential, of gastric and colon cancer cells. MKN-45 and Caco-2 were serum starved overnight, fluorescently labeled with CFSE, and plated on FluorBlok™ plates with 8 micron pores and serum free media in the top of the wells. The bottom wells were filled with serum free media, serum free media containing 10 ng/ml of G-CSF or serum containing media (positive control). As indicated in FIG. 4A, G-CSF induced increased migration of both MKN-45 and Caco-2 cells. Both cell types migrated through the pores toward serum as a positive control, and at even higher rates toward G-CSF. Up to a 4-fold increased migration was seen with G-CSF compared to serum free media, and 1.5-fold increased migration was seen with G-CSF compared to serum containing media. To further examine the potential for G-CSF to enhance the migratory ability of GI tumor cells, supernatants from tumor-derived gastric and colon MF were placed in the bottom wells and compared to wells with supernatants from normal tissue-derived MF and fresh media with serum. In FIG. 4B, supernatants from tumor-derived MF increased migration of MKN-45 and Caco-2 cells by at least 2-fold compared to normal MF supernatants. This effect was decreased by neutralizing G-CSF with monoclonal antibodies. Since MFs represent a major component of tumor stroma, their production of G-CSF could be very significant in inducing carcinoma cell mobilization and contributing to the invasive potential of the tumor.

In order to support this data, the presence of G-CSF and G-CSFR in human gastric and colon tumors was analyzed by comparing primary tumor samples from lymph node negative vs lymph node positive individuals. FIGS. 4C-F indicate that the majority of samples from node positive individuals were higher in both G-CSF and G-CSFR expression than node negative samples. For gastric tumors, G-CSF mRNA had a mean of 3.48-fold increase in samples from node negative individuals and 14.13 in samples from node positive individuals. G-CSFR mRNA was increased by 5.25-fold in samples from node negative individuals and 16.27 with node positive individuals. Similar results were seen with colon tumors where G-CSF mRNA levels were increased by 4.17-fold in tissues from node negative individuals and 17.97 in tissues from node positive individuals, while G-CSFR mRNA levels were increased by 3.62-fold in node negative and 10.56-fold in tissues from node positive individuals. These results suggest that increased G-CSF in human colon and gastric cancers is linked to cell migration from primary tumors from gastric and colon cancer patients in which cancer cells have migrated to a lymph node.

### G-CSF Expands a Population of Carcinoma Cells Expressing Stem-like Markers:

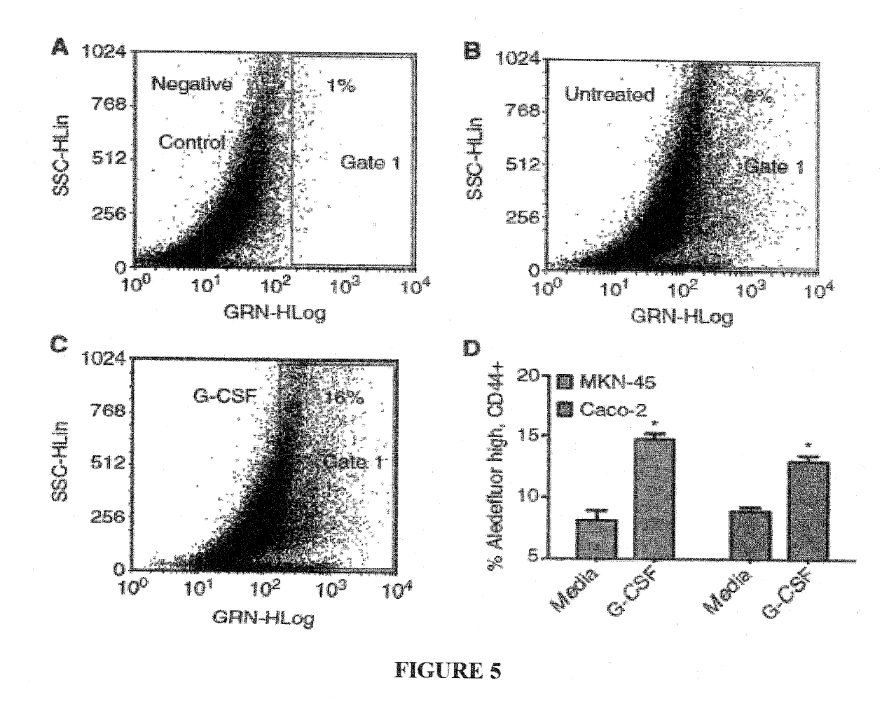

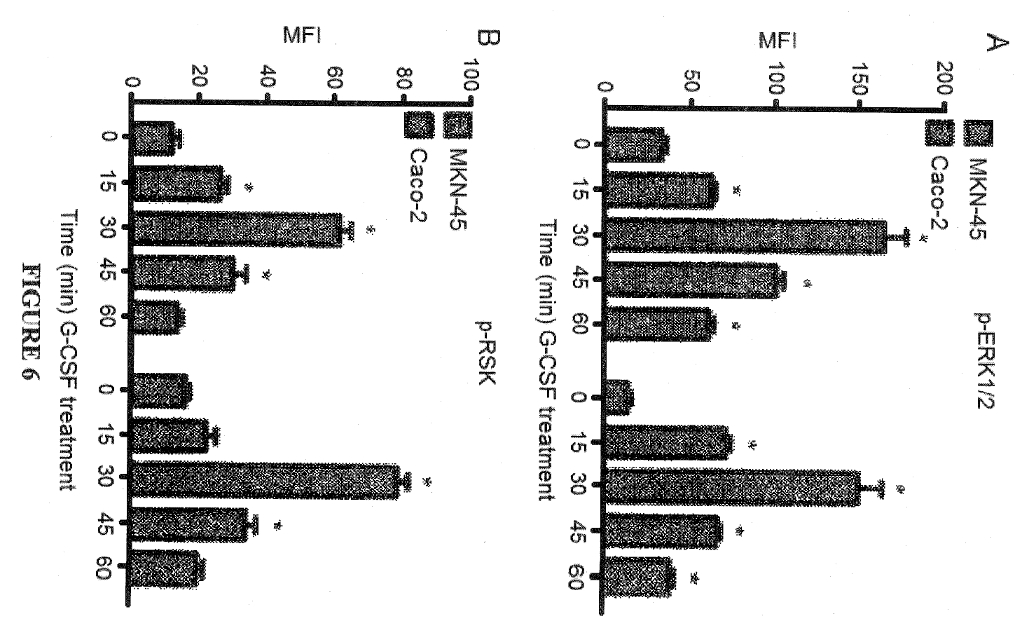

Recently, sub-populations of cells within gastrointestinal cell lines have been shown to express stem markers such as CD44 and aldehyde dehydrogenase (Huang et al., 2009; Su et al., 2011b;Takaishi et al., 2009). Therefore, assessment of MKN-45 and Caco-2 cells for a similar population was undertaken. Cells were stained with Aldefluor, which has been shown to identify cells with stem-like characteristics that stain for aldehyde dehydrogenase (Huang et al., 2009; Katsuno et al., 2012). Cells were also stained with CD44, since in gastric and colon cancers CD44 has also been shown on cells with stem-like features (Huang et al., 2009; Katsuno et al., 2012; Su et al., 2011b; Takaishi et al., 2009; Wakamatsu et al., 2012). To examine Aldefluor staining, cells were gated on the CD44 positive population. Then, another gate was set using the diethylaminobenzaldehyde (DEAB) inhibitor of aldehyde dehydrogenase as a negative control (FIG. 5A). MKN-45 cells were found to contain stem-like populations as identified by Aldefluor when compared to cells treated with DEAB (FIGS. 5A and B). G-CSF treatment expanded this population from 6% to 16% of the cell population (FIGS. 5C). Further, 48 hour treatment of MKN-45 and Caco-2 cells with 10 ng/ml of G-CSF led to an approximately 10% increase in the stem-like Aldefluor^{high}CD44⁺population (FIG. 5D) with both cell types. These data suggest that G-CSF may not only expand the numbers of hematopoietic stem cells, but also cells that express markers that may indicate stem-like characteristics.

### G-CSF Induces Proliferation Through ERK1/2 and RSK1 Signaling:

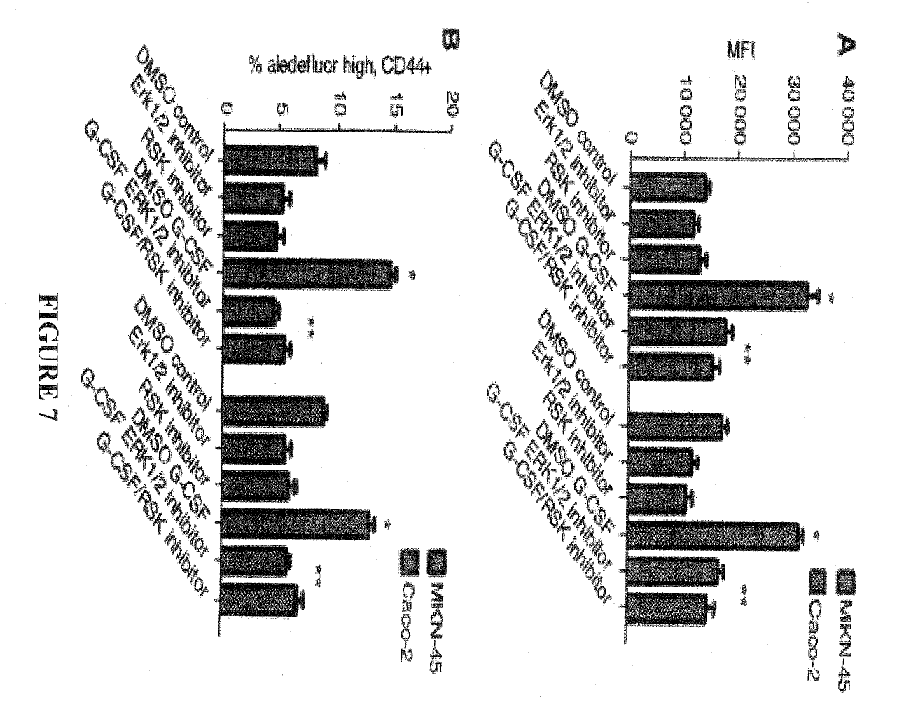

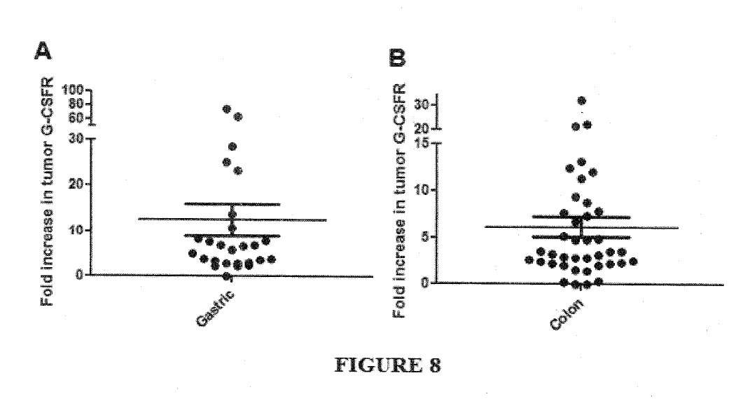

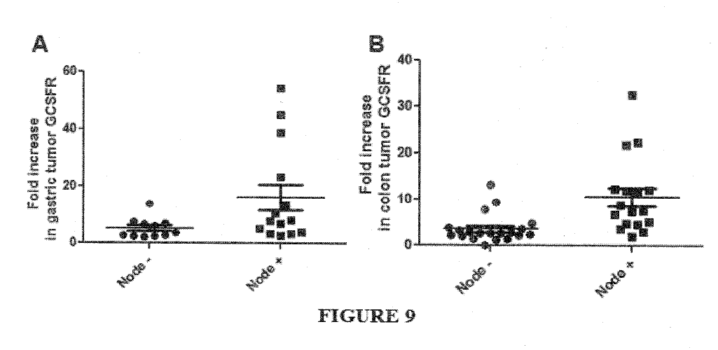

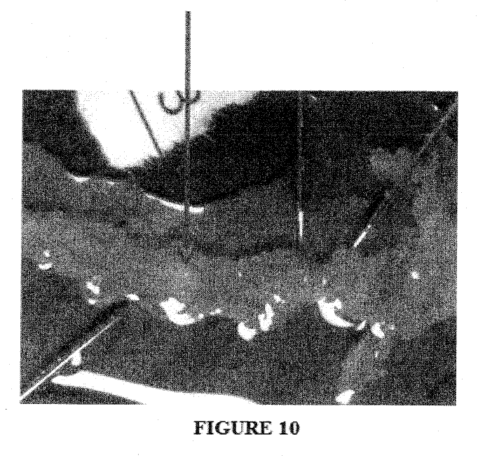

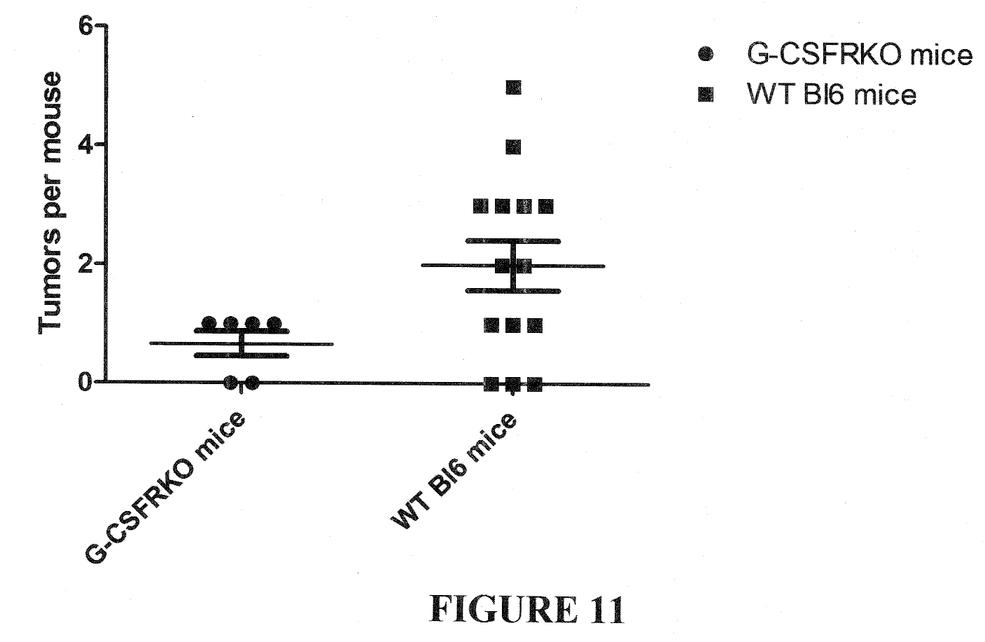

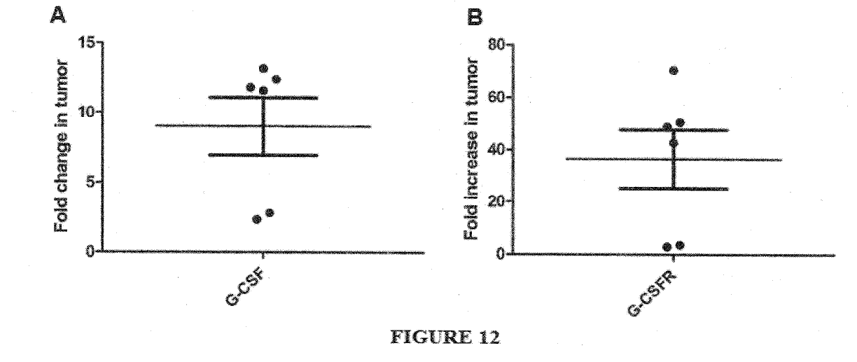

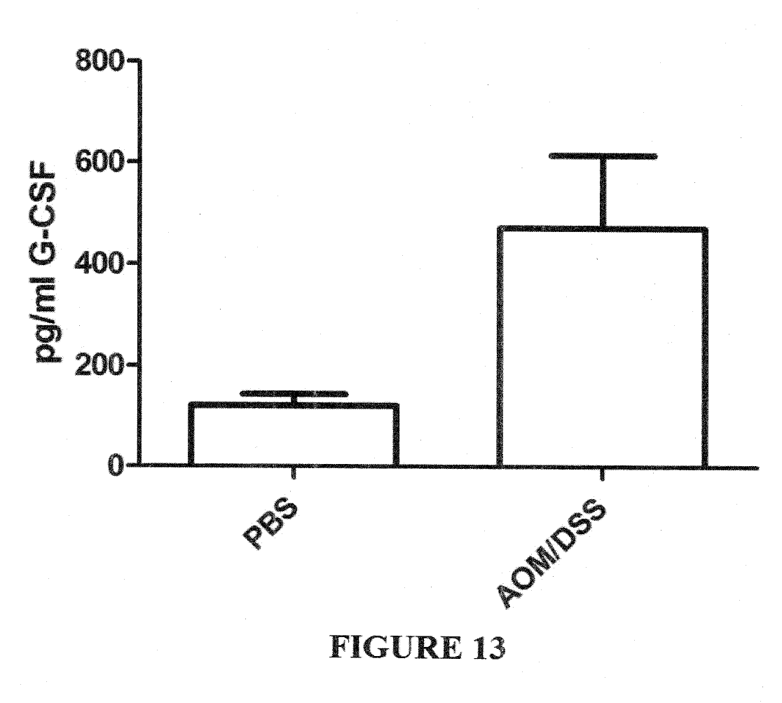

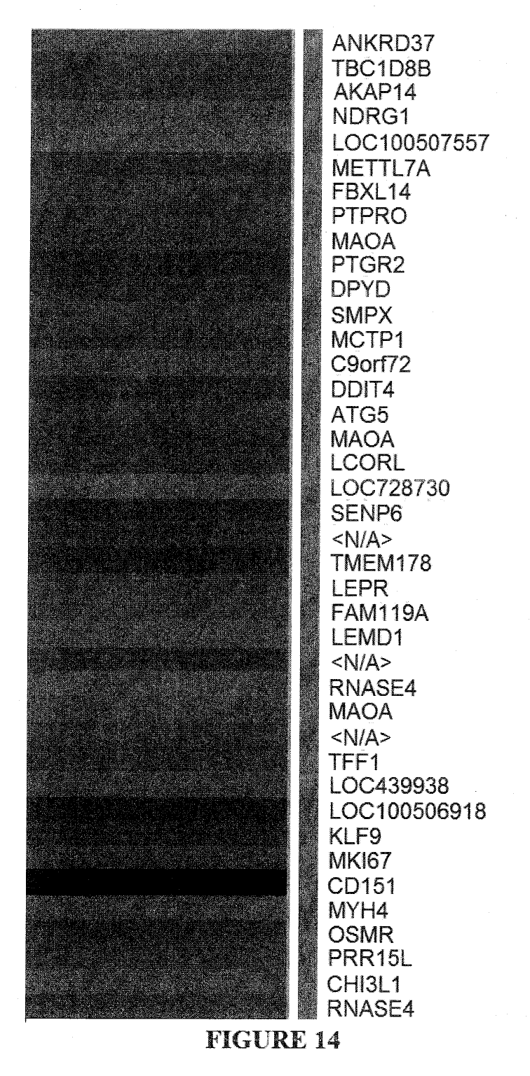

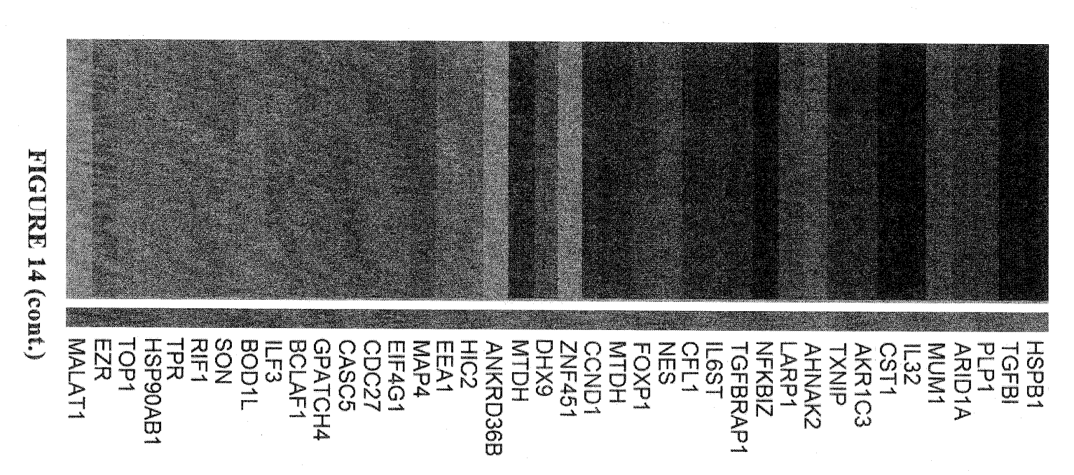

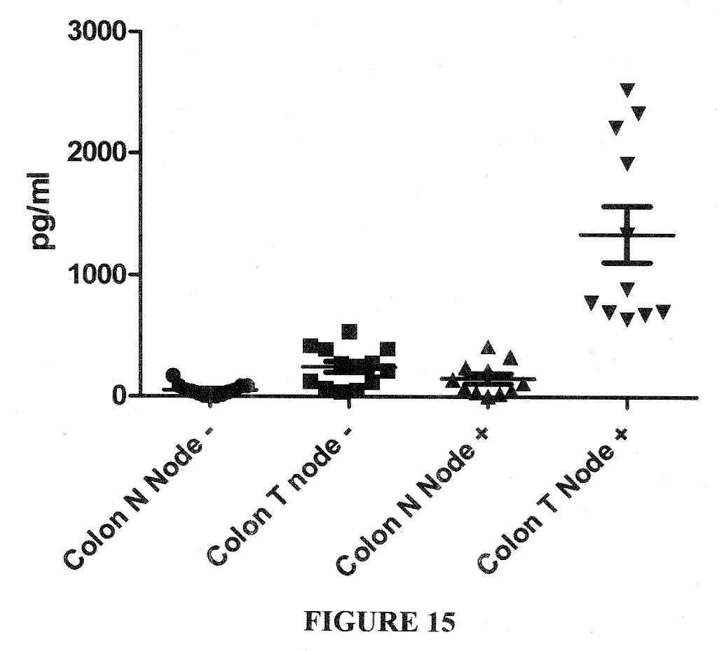

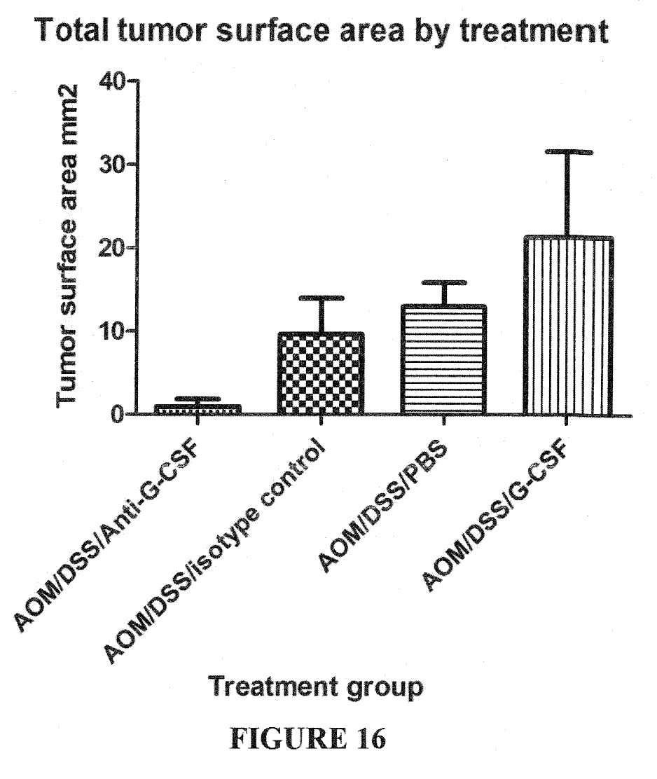

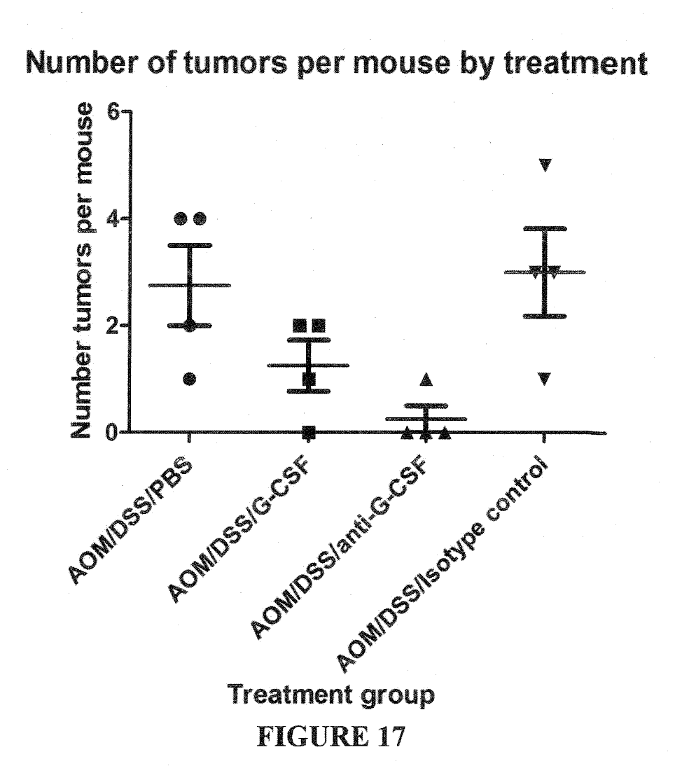

Some studies have shown that G-CSFR signaling induces signaling pathways that lead to cell proliferation (Kamezaki et aL, 2005; Wang et al., 2008). However, most of the studies have been performed in myeloid cells or neutrophils. Thus, we sought to examine G-CSFR signaling in MKN-45 and Caco-2 cells. Cells were pre-incubated with serum free media for 2 hours before treatments and recombinant G-CSF (10 ng/ml) was added to wells in 15 min intervals. Cells were lysed and protein levels normalized. Lysates were run on Luminex™ signaling bead arrays where ERK1/2 and RSK1 were found to be phosphorylated, peaking at 30 min of incubation time. A 5-fold increase in phosphorylation was found in MKN-45 and a 10-fold increase in phosphorylation was found in Caco-2 cells (FIGS. 6A and B). The other signaling molecules tested showed no change in phosphorylation after G-CSF treatment. Since we found phosphorylation of these signaling molecules after G-CSF treatment, we further sought to characterize the role of these pathways in the increased cell proliferation resulting from G-CSF treatment observed in MKN-45 and Caco-2 carcinoma cells shown in FIG. 3. ERK1/2 or RSK inhibitors were added to some wells of cells before addition of G-CSF, and proliferation examined by CyQuant proliferation assay for DNA content. FIG. 7A indicates that inhibiting either ERK1/2 or RSK drastically inhibited G-CSF induced proliferation, back to basal levels or below, indicating a mechanism of G-CSF-induced proliferation of cancer epithelial cells.

In parallel, ERK1/2 and RSK inhibition were also examined for effects on stem-like populations. Similar to the general decrease in gastric and colon carcinoma cell proliferation observed in FIG. 7A, inhibiting ERK1/2 or RSK also decreased the G-CSF-induced expansion of the stem marker expressing cells as indicated FIG. 7B when the population expressing high levels of aldehyde dehydrogenase and CD44 was examined. The G-CSF-induced stem marker expressing cell expansion was completely abrogated by treatment with either ERK1/2 or RSK inhibitors, indicating activation of these signaling pathways is key for the tumor stimulatory effects observed after G-CSF treatment.

### Discussion:

Since Virchow initially hypothesized a link between inflammation and cancer in 1863 (Balkwill and Mantovani, 2001), possible underlying mechanisms have been investigated, yet still not fully understood. G-CSF is a cytokine known to be involved in multiple cell survival, proliferation, and invasion related pathways, but its role in GI cancers has not been examined. The data presented here are similar to the findings for other tumor types in that we found increased amounts of G-CSF and G-CSFR in gastric and colorectal cancer specimens as well as in epithelial cells isolated from these human tumors. These results, along with reports in the literature of significant changes in the expression of over 300 genes, including increases in such pro-tumorigenic factors as VEGF and TGF-β after administration of G-CSF (Amariglio et al., 2007; Fujii et al., 2004; McGuire et al., 2001), led us to further investigate the potential for pro-tumorigenic effects of G-CSF on gastric and colorectal cancers. The data presented here suggest that G-CSF treatment may promote gastrointestinal tumor growth, since it induces both the proliferation and migration of gastric and colon carcinoma cells. Our data are in agreement with the previous findings from the head and neck cancer field, where it has been demonstrated that G-CSF stimulates proliferation and migration of squamous carcinoma cells (Gutschalk et al., 2006). Further, using xenograft animal models, the same research group demonstrated that G-CSF expressing tumors exhibit higher invasive capacity.

The production of G-CSF by both tumor-derived epithelial cells and fibroblasts/myofibroblasts, and epithelial expression of G-CSFR suggests autocrine and paracrine loops leading to stimulation of GI carcinoma progression. The data shown here provide a potential mechanistic link between chronic inflammation and progression of GI cancers that has not yet been considered. These data suggest that tumor derived fibroblasts/myofibroblasts respond to these malignant changes with increased secretion of G-CSF, thus enabling accelerated progression of malignancy. The observation of increased expression of both cytokine and receptor in higher tumor stage and nodal stage suggests a role for G-CSF in the progression and metastasis of human gastrointestinal cancers. Interestingly, we found the highest ligand/receptor expression in T3 stage tumors, suggesting this cytokine may confer an advantage to tumors as far as migration and tumor progression. Elevated levels of G-CSF could facilitate this process in multiple ways illustrated by the findings of this study. Increased proliferation allows for more tumor heterogeneity, supporting the development of more invasive mutations within the tumor population. Increased migration towards elevated levels of G-CSF within the stroma could assist in initiating tumor cell mobilization needed for metastasis. Thus, it is possible that suppression of this cytokine axis within tumors may decrease metastatic potential once cancers are diagnosed, or perhaps reverse progression from dysplasia to carcinoma in high risk patients. Clearly, further investigation into the role of this cytokine in human gastrointestinal cancers is warranted.

G-CSF is most well-known for its ability to stimulate migration of bone marrow derived granulocyte precursor stem cells, which results in increased white blood cell counts. This led us to consider its possible effects on cells that express stem-like markers since it has been postulated that cancer is a disease initiated and maintained by stem cells (Jiang et al., 2012). A substantial body of evidence supports the hypothesis that neoplasms are initiated and maintained by a small population of cells within a tumor that possess properties similar to those of normal adult stem cells (Lobo et al., 2007). These qualities include the ability to self-renew and generate differentiated progeny. According to this hypothesis, only a small subset of tumor cells are required to initiate and sustain malignant tumor growth and to give rise to the phenotypic heterogeneity observed in the original tumor; this is true for a wide variety of cancers, including gastric and colorectal cancer (Dalerba et al., 2007; Singh, 2013). Although markers of stem-like cells are debated in the field, many groups agree that high expression of aldehyde dehydrogenase is a strong indicator of stem-like characteristics (Huang et al., 2009). Additionally, several studies have indicated that CD44 is highly expressed by cells with stem-like characteristics in both gastric and colon cancers (Takaishi et al., 2009; Wakamatsu et al., 2012; Wang et al., 2011). Thus, we chose to examine the effects of G-CSF on the subset of cells expressing these markers within gastric and colon carcinoma cells. Cells expressing these markers were found to be increased in number after G-CSF treatment. Similar data were found for prostate cancer (Ma et al., 2012), wherein stimulation with G-CSF of prostate cancer cells resulted in an increase in the number of cells expressing prostate cancer stem markers. Taken together with this report, our data suggest that G-CSF treatment may promote maintenance of higher levels of cells expressing markers representing stem-like characteristics. These findings raise additional concern about G-CSF in gastric or colon cancers. Indeed, if G-CSF increases the population of cells with stem-like qualities in vitro, it is possible it could increase risk of further metastasis or resistance to therapy.

The effects of G-CSF on proliferation of GI carcinoma cells are not surprising given the activation of such key proliferation pathways. We found significant activation in both the RSK and Erk1/2 pathways in gastric and colorectal cells after treatment with G-CSF. In addition, it was confirmed that the increased proliferation and migration in our studies were due to phosphorylation of these signaling molecules. Interestingly, the increased population of cells expressing stem-like markers after treatment with G-CSF was reduced upon blockade of these pathways as well, suggesting that many of the potentially deleterious effects of G-CSF may be due to activation of RSK and ERK1/2. Others have also shown a role for G-CSF/G-CSFR in activating the JAK/STAT pathway along with the ERK pathway (Marino and Roguin, 2008; Sampson et al., 2007). In our study, we found the ERK and RSK pathways to be the major players, which may reflect a difference in cell types examined.

Given the data presented here with gastric and colon tumor production of G-CSF and the resulting induction of proliferation and expansion of carcinoma cells, there is evidence that G-CSF may be an important link between inflammation and tumor progression. Thus, there is also the potential that this receptor and cytokine could be a prognostic marker. Further examination of these pathways could lead to changes in treatment for patients with this tumor attribute.

In this study, G-CSF expression was examined in human gastric and colon tumors and by tumor-derived stromal myofibroblasts and carcinoma cells. G-CSFR expression was examined on carcinoma cells isolated from human tissues. The effects of G-CSF on gastric and colon carcinoma cell proliferation, migration and signaling were examined.

The results showed that G-CSFR was highly expressed in 90% of human gastric and colon carcinomas. G-CSF was also found to be highly produced by stromal myofibroblasts and carcinoma cells. Exposure of carcinoma cells to G-CSF led to increased proliferation and migration, and expansion of a subpopulation of carcinoma cells expressing stem-like markers. These processes were dependent on ERK1/2 and RSK1 phosphorylation.

Conclusions: These data suggest that the G-CSF/R axis promotes gastric and colorectal cancer development and suggests they are potential tumor targets in all cells which express G-CSF and G-CSF receptors, especially including at high levels.

Experimentation revealed that G-CSFR KO mice in a background of C57BL/6 treated with a carcinogen/inflammation model of colon cancer (AOM/DSS) develop distal tumors. The test parameters were as follows:

KO Group
N=6
Mean weight mouse=21.97 gms (range 21.18-23.6 gms)
5/6 mice had one tumor each (0.833 tumors/mouse)
All but one were distal 1/3 colon (exception was distal mid1/3)
Mean tumor #/mouse=1
Mean/median tumor volume=6.36 mm³/5 mm³

WT Group
N=6

Mean weight mouse 21.63 gms (range 20-23.25 gms)
4/6 mice had tumors
All tumors were proximal to mid 1/3 colon
Mean tumor #/mouse: 8 tumors/4 mice out of 6 mice
Mean/median tumor volume=7.1 mm³/6.75 mm³

### REFERENCES

Amariglio N, Jacob-Hirsch J, Shimoni A, Leiba M, Rechavi G, Nagler A (2007) Changes in gene expression pattern following granulocyte colony-stimulating factor administration to normal stem cell sibling donors. Acta Haematol117 (2): 68-73, doi:96856[pii];10.1159/000096856 [doi]
Balkwill F, Mantovani A (2001) Inflammation and cancer: back to Virchow? Lancet 357 (9255): 539-545, doi:S0140-6736 (00)04046-0[pii];10.1016/50140-6736 (00)04046-0[doi]
Carpentino J E, Hynes M J, Appelman H D, Zheng T, Steindler D A, Scott E W, Huang E H (2009) Aldehyde dehydrogenase-expressing colon stem cells contribute to tumorigenesis in the transition from colitis to cancer. Cancer Res69 (20): 8208-8215, doi:0008-5472.CAN-09-1132 [pii];10.1158/0008-5472.CAN-09-1132[doi]
Dalerba P, Cho R W, Clarke M F (2007) Cancer stem cells: models and concepts. Annu Rev Med 58 267-284, doi:10.1146/annurev.med.58.062105.204854[doi]
Deng S, Yang X, Lassus H, Liang S, Kaur S, Ye Q, Li C, Wang L P, Roby K F, Orsulic S, Connolly D C, Zhang Y, Montone K, Butzow R, Coukos G, Zhang L (2010) Distinct expression levels and patterns of stem cell marker, aldehyde dehydrogenase isoform 1 (ALDH1), in human epithelial cancers. PLoS One 5 (4): e10277, doi:10.1371/journal.pone.0010277[doi] Fujii K, Ishimaru F, Kozuka T, Matsuo K, Nakase K, Kataoka I, Tabayashi T, Shinagawa K, Ikeda K, Harada M, Tanimoto M (2004) Elevation of serum hepatocyte growth factor during granulocyte colony-stimulating factor-induced peripheral blood stem cell mobilization. Br J Haematol 124(2): 190-194, doi:4745[pii]
Gillen C D, Walmsley R S, Prior P, Andrews H A, Allan R N (1994) Ulcerative colitis and Crohn's disease: a comparison of the colorectal cancer risk in extensive colitis. Gut 35 (11): 1590-1592 Gutschalk C M, Herold-Mende C C, Fusenig NE, Mueller M M (2006) Granulocyte colony-stimulating factor and granulocyte-macrophage colony-stimulating factor promote malignant growth of cells from head and neck squamous cell carcinomas in vivo. Cancer Res 66 (16): 8026-8036, doi:66/16/8026[pii];10.1158/0008-5472.CAN-06-0158[doi]
Held T K, Gundert-Remy U (2010) Pharmacodynamic effects of haematopoietic cytokines: the view of a clinical oncologist. Basic Clin Pharmacol Toxicol 106 (3): 210-214, doi:PTO514 [pii];10.1111/j.1742-7843.2009.00514.x[doi]
Hirai K, Kumakiri M, Fujieda S, Sunaga H, Lao L M, Imamura Y, Ueda K, Fukuda M (2001) Expression of granulocyte colony-stimulating factor and its receptor in epithelial skin tumors. J Dermatol Sci 25 (3): 179-188, doi:S0923181100001316[pii]
Huang E H, Hynes M J, Zhang T, Ginestier C, Dontu G, Appelman H, Fields J Z, Wicha M S, Boman B M (2009) Aldehyde dehydrogenase 1 is a marker for normal and malignant human colonic stem cells (SC) and tracks SC overpopulation during colon tumorigenesis. Cancer Res 69 (8): 3382-3389, doi:0008-5472.CAN-08-4418[pii];10.1158/0008-5472.CAN-08-4418[doi] Jemal A, Center M M, DeSantis C, Ward E M (2010) Global patterns of cancer incidence and mortality rates and trends. Cancer Epidemiol Biomarkers Prey 19 (8): 1893-1907, doi:1055-9965. EPI-10-0437[pii];10.1158/1055-9965.EPI-10-0437[doi]
Jiang W, Peng J, Zhang Y, Cho W C, Jin K (2012) The implications of cancer stem cells for cancer therapy. Int J Mol Sci 13 (12): 16636-16657, doi:ijms131216636 [pii];10.3390/ijms131216636[doi]
Kamezaki K, Shimoda K, Numata A, Haro T, Kakumitsu H, Yoshie M, Yamamoto M, Takeda K, Matsuda T, Akira S, Ogawa K, Harada M (2005) Roles of Stat3 and ERK in G-CSF signaling. Stem Cells 23 (2): 252-263, doi:23/2/252[pii];10.1634/stemcells.2004-0173a [doi]
Katsuno Y, Ehata S, Yashiro M, Yanagihara K, Hirakawa K, Miyazono K (2012) Coordinated expression of REG4 and aldehyde dehydrogenase 1 regulating tumourigenic capacity of diffuse-type gastric carcinoma-initiating cells is inhibited by TGF-beta. J Pathol, doi:10.1002/path.4020[doi]
Liongue C, Wright C, Russell A P, Ward A C (2009) Granulocyte colony-stimulating factor receptor: stimulating granulopoiesis and much more. Int J Biochem Cell Biol 41 (12): 2372-2375, doi:S1357-2725 (09)00233-7[pii];10.1016/j.bioce1.2009.08.011[doi]
Lobo N A, Shimono Y, Qian D, Clarke M F (2007) The biology of cancer stem cells. Annu Rev Cell Dev Biol 23 675-699, doi:10.1146/annurev.cellbio.22.010305.104154[doi]
Ma Y, Liang D, Liu J, Axcrona K, Kvalheim G, Giercksky K E, Nesland J M, Suo Z (2012) Synergistic effect of SCF and G-CSF on stem-like properties in prostate cancer cell lines. Tumour Biol 33 (4): 967-978, doi:10.1007/s13277-012-0325-3[doi]
Marino V J, Roguin L P (2008) The granulocyte colony stimulating factor (G-CSF) activates Jak/STAT and MAPK pathways in a trophoblastic cell line. J Cell Biochem 103 (5): 1512-1523, doi:10.1002/jcb.21542[doi]
McGuire T R, Kessinger A, Hock L, Sharp J G (2001) Elevated transforming growth factor beta levels in the plasma of cytokine-treated cancer patients and normal allogeneic stem cell donors. Cytotherapy 3 (5): 361-364, doi:10.1080/146532401753277652[doi]
Morstyn G, Campbell L, Lieschke G, Layton J E, Maher D, O′Connor M, Green M, Sheridan W, Vincent M, Alton K, . (1989) Treatment of chemotherapy-induced neutropenia by subcutaneously administered granulocyte colony-stimulating factor with optimization of dose and duration of therapy. J Clin Oncol 7 (10): 1554-1562
Ninci E B, Brandstetter T, Meinhold-Heerlein I, Bettendorf H, Sellin D, Bauknecht T (2000) G-CSF receptor expression in ovarian cancer. Int J Gynecol Cancer 10 (1): 19-26, doi:ijg99076[pii]
Ohata H, Ishiguro T, Aihara Y, Sato A, Sakai H, Sekine S, Taniguchi H, Akasu T, Fujita S, Nakagama H, Okamoto K (2012) Induction of the stem-like cell regulator CD44 by Rho kinase inhibition contributes to the maintenance of colon cancer-initiating cells. Cancer Res 72 (19): 5101-5110, doi:0008-5472.CAN-11-3812[pii];10.1158/0008-5472.CAN-11-3812[doi]
Pinchuk I V, Morris K T, Nofchissey R A, Earley R B, Wu J Y, Ma T Y, Beswick E J (2013) Stromal Cells Induce Th17 during Helicobacter pylori Infection and in the Gastric Tumor Microenvironment. PLoS One 8 (1): e53798, doi:10.1371/journal.pone.0053798[doi];PONE-D-12-31285[pii]
Saada J I, Pinchuk I V, Barrera C A, Adegboyega P A, Suarez G, Mifflin R C, Di Mari J F, Reyes V E, Powell D W (2006) Subepithelial myofibroblasts are novel nonprofessional APCs in the human colonic mucosa. J lmmunol 177 (9): 5968-5979
Sampson M, Zhu Q S, Corey S J (2007) Src kinases in G-CSF receptor signaling. Front Biosci 12 1463-1474, doi:2160[pii]
Seelentag W, Mermod J J, Vassalli P (1989) Interleukin 1 and tumor necrosis factor-alpha additively increase the levels of granulocyte-macrophage and granulocyte colony-stimulating factor (CSF) mRNA in human fibroblasts. Eur Jlmmunol 19 (1): 209-212, doi:10.1002/eji.1830190135[doi]
Singh S R (2013) Gastric cancer stem cells: A novel therapeutic target. Cancer Lett, doi:S0304-3835 (13)00278-4[pii];10.1016/j.canlet.2013.03.035[doi]
Su Y J, Lai H M, Chang Y W, Chen G Y, Lee J L (2011a) Direct reprogramming of stem cell properties in colon cancer cells by CD44. EMBO J 30 (15): 3186-3199, doi:emboj2011211[pii];10.1038/emboj.2011.211[doi]
Su Y J, Lai H M, Chang Y W, Chen G Y, Lee J L (2011b) Direct reprogramming of stem cell properties in colon cancer cells by CD44. EMBO J30 (15): 3186-3199, doi:emboj2011211[pii];10.1038/emboj.2011.211[doi]
Sullivan J P, Spinola M, Dodge M, Raso M G, Behrens C, Gao B, Schuster K, Shao C, Larsen J E, Sullivan L A, Honorio S, Xie Y, Scaglioni P P, DiMaio J M, Gazdar A F, Shay J W, Wistuba I I, Minna J D (2010) Aldehyde dehydrogenase activity selects for lung adenocarcinoma stem cells dependent on notch signaling. Cancer Res 70 (23): 9937-9948, doi:0008-5472.CAN-10-0881[pii];10.1158/0008-5472.CAN-10-0881[doi]
Sunaga H, Fujieda S, Tsuzuki H, Asamoto K, Fukuda M, Saito H (2001) Expression of granulocyte colony-stimulating factor receptor and platelet-derived endothelial cell growth factor in oral and oropharyngeal precancerous lesions. Anticancer Res 21 (4B): 2901-2906
Tachibana M, Murai M (1998) G-CSF production in human bladder cancer and its ability to promote autocrine growth: a review. Cytokines Cell Mol Ther 4 (2): 113-120 p0 Takaishi S, Okumura T, Tu S, Wang S S, Shibata W, Vigneshwaran R, Gordon S A, Shimada Y, Wang T C (2009) Identification of gastric cancer stem cells using the cell surface marker CD44. Stem Cells 27 (5): 1006-1020, doi:10.1002/stem.30[doi]
Torpy J M, Lynm C, Glass R M (2010) JAMA patient page. Stomach cancer. JAMA 303 (17): 1771, doi:303/17/1771[pii];10.1001/jama.303.17.1771[doi]
Wakamatsu Y, Sakamoto N, Oo H Z, Naito Y, Uraoka N, Anami K, Sentani K, Oue N, Yasui W (2012) Expression of cancer stem cell markers ALDH1, CD44 and CD133 in primary tumor and lymph node metastasis of gastric cancer. Pathol Int 62 (2): 112-119, doi:10.1111/j.1440-1827.2011.02760.x[doi]
Wang L, Xue J, Zadorozny E V, Robinson L J (2008) G-CSF stimulates Jak2-dependent Gab2 phosphorylation leading to Erk1/2 activation and cell proliferation. Cell Signal 20 (10): 1890-1899, doi:S0898-6568 (08)00194-0[pii];10.1016/j.cellsig.2008.06.018[doi]
Wang T, Ong C W, Shi J, Srivastava S, Yan B, Cheng C L, Yong W P, Chan S L, Yeoh K G, Iacopetta B, Salto-Tellez M (2011) Sequential expression of putative stem cell markers in gastric carcinogenesis. Br J Cancer 105 (5): 658-665, doi:bjc2011287[pii];10.1038/bjc.2011.287[doi]
Worthley D L, Giraud A S, Wang TC (2010) Stromal fibroblasts in digestive cancer. Cancer Microenviron 3 (1): 117-125, doi:10.1007/s12307-009-0033-8[doi]
Yokoyama T, Hyodo M, Hosoya Y, Koinuma K, Kurashina K, Saitoh S, Hirashima Y, Arai W, Zuiki T, Yasuda Y, Nagai H, Kanai N, Saitoh K (2005) Aggressive G-CSF-producing gastric cancer complicated by lung and brain abscesses, mimicking metastases. Gastric Cancer 8 (3): 198-201, doi:10.1007/s10120-005-0335-6[doi]

1. A pharmaceutical composition comprising a therapeutically effective amount of at least one G-CSF inhibitor, in combination with at least one additional anticancer agent.

2. The composition according to claim 1 wherein said additional anticancer is a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody or a mixture thereof.

3. The composition according to claim 1 wherein said additional anticancer agent is verolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1 H -pyrrolo[2,3-d ]pyrimidin-5-ypethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)- indolylj-quinolone, vatalanib, AG-013736, AVE-0005, the acetate salt of [D-Ser(Bu t) 6,Azgly 10] (pyro-Glu-His-Trp-Ser-Tyr-D-Ser(Bu t)-Leu-Arg-Pro-Azgly-NH ₂ acetate [C₅₉H₈₄N₁₈Oi₄-(C₂H₄O₂) _{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol., vairubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa or a mixture thereof.

4. The composition according to claim 1 wherein said additional anticancer agent is at least one agent selected from the group consisting of mechlorethamine (nitrogen mustard), chlorambucil, cyclophosphamide, ifosfamide, melphalan; nitrosoureas, including streptozocin, carmustine (BCNU), and lomustine; alkyl sulfonates, including busulfan; Triazines, including dacarbazine (DTIC) and temozolomide; ethylenimines, including thiotepa and altretamine (hexamethylmelamine); platinum drugs, including cisplatin, carboplatin and oxalaplatin; Antimetabolites including fluorouracil (5-FU), 6-mercaptopurine (6-MP), Capecitabine, Cladribine, Clofarabine, Cytarabine, Floxuridine, Fludarabine, Gemcitabine, Hydroxyurea, Methotrexate, Pemetrexed, Pentostatin, Thioguanine; Anti-tumor antibiotics including Anthracyclines, such as Daunorubicin, Doxorubicin, Epirubicin, Idarubicin and non-anthracycline antibioitics Actinomycin-D , Bleomycin and Mitomycin-C; Topoisomerase inhibitors including topotecan and irinotecan (CPT-11), etoposide (VP-16), teniposide and Mitoxantrone; Mitotic inhibitors, including Taxanes: paclitaxel and docetaxel; Epothilones, including ixabepilone; Vinca alkaloids, including vinblastine , vincristine, and vinorelbine, Estramustine; and Targeted therapies including imatinib, gefitinib, sunitiniband and bortezomib.

5. A method of treating cancer in a patient in need comprising administering to said patient comprising administering an effective amount of a pharmaceutical composition comprising a G-CSF inhibitor or a mixture thereof, optionally in combination with an additional anticancer agent.

6. A method of reducing the likelihood that a cancer will metastasize in a patient with cancer or recur in a patient whose cancer is in remission, the method comprising administering to said patient an effective amount of a pharmaceutical composition comprising a G-CSF inhibitor or a mixture thereof, optionally in combination with an additional anticancer agent.

7. The method according to claim 5 wherein said G-CSF inhibitor is a compound that binds to G-CSF.

8. The method according to claim 5 wherein said G-CSF inhibitor is a compound that binds to the G-CSF receptor.

9. The method according to claim 5 wherein said G-CSF inhibitor is a small molecule that inhibits the binding of G-CSF to the G-CSF receptor.

10. The method according to claim 5 wherein said cancer is metastatic cancer.

11. The method according to any claim 5 wherein said cancer is recurrent cancer.

12. The method according to claim 5 wherein said G-CSF inhibitor is an anti-G-CSF antibody.

13. The method according to claim 12 wherein said antibody is anti-human G-CSF monoclonal antibody clone 4A4RC .

14. The method according to claim 8 wherein said inhibitor is an anti-human G-CSF receptor antibody.

15. The method according to claim 5 wherein said cancer is a carcinoma, a leukemia; lymphoma, melanoma; myeloproliferative disease; sarcoma, a tumor of the central nervous system, a germ-line tumor; a mixed types of neoplasia, particularly carcinosarcoma and Hodgkin's disease; and tumors of mixed origin, such as Wilms′ tumor and teratocarcinomas.

16. The method according to claim 5 wherein said cancer is a squamous-cell carcinoma, adenocarcinoma, hepatocellular carcinoma, renal cell carcinomas, bladder cancer, bowel cancer, breast cancer, cervical cancer, colon (colorectal) cancer, esophageal cancer, head cancer, kidney cancer, liver cancer, thyroid cancer, lung cancer, neck cancer, ovary (ovarian) cancer, pancreatic cancer, prostate cancer, stomach cancer;
leukemia, lymphoma, melanoma; myeloproliferative disease; Ewing's sarcoma, hemangiosarcoma, Kaposi's sarcoma, liposarcoma, myosarcomas, peripheral neuroepithelioma, and synovial sarcoma; glioma, astrocytoma, oligodendroglioma, ependymoma, gliobastoma, neuroblastoma, ganglioneuroma, ganglioglioma, medulloblastoma, pineal cell tumor, meningioma, meningeal sarcoma, neurofibroma, Schwannoma, uterine cancer, lung cancer, testicular cancer, thyroid cancer, astrocytoma, stomach cancer, Wilms′ tumor and teratocarcinoma.

17. The method according to claim 5 wherein said inhibitor is administered in combination with an additional anticancer agent.

18. The method according to claim 17 wherein said additional anticancer agent is a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody or a mixture thereof.

19. The method according to claim 17 wherein said additional anticancer agent is verolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1 H-pyrrolo[2,3-d ]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,);3[5-(methylsulfonylpiperadinemethyl)-indolylj-quinolone, vatalanib, AG-013736, AVE-0005, the acetate salt of [D-Ser(Bu t) 6,Azgly 10] (pyro-Glu-His-Trp-Ser-Tyr-D-Ser(Bu t)-Leu-Arg-Pro- Azgly-NH ₂ acetate [C₅₉H₈₄N₁₈Oi₄-(C₂H₄O₂) x where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa or a mixture thereof.

20. The method according to claim 17 wherein said additional anticancer agent is at least one agent selected from the group consisting of mechlorethamine (nitrogen mustard), chlorambucil, cyclophosphamide, ifosfamide, melphalan; nitrosoureas, including streptozocin, carmustine (BCNU), and lomustine; alkyl sulfonates, including busulfan; Triazines, including dacarbazine (DTIC) and temozolomide; ethylenimines, including thiotepa and altretamine (hexamethylmelamine); platinum drugs, including cisplatin, carboplatin and oxalaplatin;
Antimetabolites including fluorouracil (5-FU), 6-mercaptopurine (6-MP), Capecitabine, Cladribine, Clofarabine, Cytarabine, Floxuridine, Fludarabine, Gemcitabine, Hydroxyurea, Methotrexate, Pemetrexed, Pentostatin, Thioguanine; Anti-tumor antibiotics including Anthracyclines, such as Daunorubicin, Doxorubicin, Epirubicin, Idarubicin and non-anthracycline antibioitics Actinomycin-D , Bleomycin and Mitomycin-C; Topoisomerase inhibitors including topotecan and irinotecan (CPT-11), etoposide (VP-16), teniposide and Mitoxantrone; Mitotic inhibitors, including Taxanes: paclitaxel and docetaxel; Epothilones, including ixabepilone; Vinca alkaloids, including vinblastine , vincristine, and vinorelbine, Estramustine; and Targeted therapies including imatinib, gefitinib, sunitiniband and bortezomib.

21. The method according to claim 5 which is combined with radiation therapy.

22. The method according to claim 5 which is combined with at least one alternative anticancer therapy selected from the group consisting of hormonal therapy, proton therapy, cryosurgery, and/or high intensity focused ultrasound (HIFU), radiofrequency ablation, microwave ablation, transarterial therapies such as radioembolization with Y90 or bland embolization and chemoembolization.
